(19) United States
(12) Patent Application Publication
(10) Pub. No.: US 2026/0224533 A1
(43) Pub. Date: Aug. 6, 2026

(54) CRYSTALLINE NANOPARTICLES COMPRISING ENZALUTAMIDE

(71) Applicants: HELM PHARMACEUTICALS GMBH, Hamburg (DE); Nanoform Finland Oyj, Helsinki (FI)

(72) Inventors: Martti KAASALAINEN, Helsinki (FI); Heiko BRUNNER, Quickborn (DE); Tatiana DANILOVA, Helsinki (FI); Amruta GORAJIYA, Helsinki (FI); Niklas SANDLER, Helsinki (FI); Virginia NYKÄNEN, Helsinki (FI)

(21) Appl. No.: 19/148,303

(22) PCT Filed: Jan. 18, 2024
(86) PCT No.: PCT/EP2024/051122

§ 371 Date: Jul. 15, 2025

(30) Foreign Application Priority Data
Jan. 18, 2023 (EP) 23152127.9

(51) Int. Cl. A61K 31/4166; A61K 9/51

(52) U.S. Cl. CPC A61K 31/4166; A61K 9/5138; A61K 9/5192

(57) ABSTRACT

The invention relates to a composition comprising or essentially consisting of (i) nanoparticles comprising or essentially consisting of Enzalutamide in crystalline form and (ii) one or more physiologically acceptable polymers and/or copolymers. The invention also relates to processes for the preparation of such compositions, pharmaceutical dosage forms comprising or made from such compositions, and uses of such pharmaceutical dosage forms for medical purposes.

Priority is claimed of European patent application no. 23 152 127.9 that was filed on Jan. 18, 2023.

The invention relates to a composition comprising or essentially consisting of (i) nanoparticles comprising or essentially consisting of Enzalutamide in crystalline form and (ii) one or more physiologically acceptable polymers and/or copolymers. The invention also relates to processes for the preparation of such compositions, pharmaceutical dosage forms comprising or made from such compositions, and uses of such pharmaceutical dosage forms for medical purposes.

Enzalutamide is an androgen receptor signaling inhibitor used as an agent for treating castration-resistant prostate cancer (U.S. Pat. No. 7,709,517). Enzalutamide is a BCS Class II drug, i.e. exhibits high permeability but low solubility. Enzalutamide is provided commercially as soft gel capsules and tablets (brand name “XTANDI®”). The soft gel capsules are filled with a liquid comprising 40 mg of Enzalutamide per one capsule and pharmaceutical excipients. Tablets comprise 40 or 80 mg Enzalutamide per one tablet and pharmaceutical excipients. The daily dosage is typically 160 mg, and a patient therefore needs to take four capsules or four 40 mg tablets or two 80 mg tablets daily. A suitable single tablet of reasonable size comprising the prescribed amount of Enzalutamide and having suitable and advantageous solubility and/or dissolution stability and absorption would be advantageous as a suitable alternative to commercially available soft gel capsules and tablets. Such a tablet should provide increased patient compliance and lower incidence of dosing errors.

WO 2014/043208 A1 provides formulations of Enzalutamide and their use for treating hyperproliferative disorders.

US 2015/0239848 A1 relates to Enzalutamide polymorphic forms and the preparation thereof.

V. Wilson et al., Journal of Controlled Release, 292 (2018) 172-182 relates to amorphous solid dispersions of Enzalutamide that are prepared with hydrophilic polymers hydroxypropyl methyl cellulose acetate succinate and copovidone (PVP/VA). The formulations were tested in vivo in rats using oral dosing of amorphous solid dispersions. Amorphous solid dispersions that underwent crystallization showed lower plasma exposures. Differences were also observed between amorphous solid dispersions that dissolved to form nanosized amorphous drug aggregates versus those that dissolved to yield only supersaturated solutions, with the former outperforming the latter in terms of the plasma exposure. The authors conclude that these observations highlight the importance of thoroughly understanding the phase behavior of an amorphous formulation following dissolution and the need to discriminate between different types of precipitation, specifically crystallization versus glass liquid phase separation to form nanosized amorphous aggregates.

Methods of producing microparticles and nanoparticles are described in various patent applications and patents, for example in U.S. Pat. Nos. 5,833,891, 5,534,270, 6,862,890, 6,177,103, DE 10 2005 053 862, U.S. Pat. Nos. 5,833,891, 5,534,270, 6,862,890, 6,177,103, DE 10 2005 017 777 and DE 10 2005 053 862.

Ch. Thangavel et al., Mol. Pharm. 2018, 15 (5), 1778-1790 relates to anti-PSMA-conjugated hybrid antiandrogen nanoparticles and their therapeutic efficacy and cellular toxicity.

WO 02/060275 A1 describes methods of producing nanoparticles in which two immiscible liquids are charged electrically so as to achieve encapsulation. In this case, the use of toxic substances is not ruled out, meaning that product quality may suffer considerably as a result. Particle size, moreover, cannot be controlled with this method.

US 2009/0214655 A1 also describes the use of two immiscible liquids. Although a microreactor is used there to produce the nanoparticles, only the production of emulsions is described. In addition, the nanoparticles are produced in a liquid-filled space in which, once again, it is impossible to control either particle size or the particle properties. Furthermore, the device can easily become blocked due to the fact that the reactions are carried out in microchannels.

Dissolution rate of an active pharmaceutical ingredient generally depends on the available surface area. Particle size reduction can be used to increase the surface area and the dissolution rate; it may additionally increase solubility. Solubility may also be increased by providing the active pharmaceutical ingredient in amorphous form. Therefore, particle size reduction and amorphization are valid approaches for improving the bioavailability of active pharmaceutical ingredient with low solubility. Unfortunately, amorphous materials tend to crystallize during processing, storage or administration. Crystallization may also detrimentally increase the particle size.

WO 2014/041487 A2 relates to crystalline and amorphous forms of Enzalutamide. The present application further relates to amorphous solid dispersions of Enzalutamide with pharmaceutically acceptable carriers. The present application also relates to processes for the preparation of crystalline forms R1 and R2, amorphous form and amorphous solid dispersion of Enzalutamide.

WO 2019/030691 A1 relates to an oral extrudate pharmaceutical compositions of Enzalutamide. The extrudate compositions comprise one or more suitable polymers and are prepared using twin screw extrusion or hot melt extrusion. Methods of preparing such compositions are also provided. The extrudate compositions may be used for the treatment of prostate cancer.

WO 2020/234448 A1 relates to nanoparticles comprising Enzalutamide, processes for the preparation of such nanoparticles, pharmaceutical compositions and pharmaceutical dosage forms comprisesing such nanoparticles, processes for the preparation of such pharmaceutical dosage forms, and uses of the pharmaceutical dosage forms for medical purposes.

Controlled Expansion of Supercritical Solutions (CESS™) is a known process for producing nanoparticles of pharmacologically active ingredients. The CESS™ process was developed by Prof. Jouko Yliruusi and Prof. Edward Hæggström at the University of Helsinki and Nanoform Finland Oyj. The controlled process produces small and uniformly sized particles that are made directly from solution without excipients. For details, reference can be made to e.g. J. Pessi et al., Controlled Expansion of Supercritical Solution: A Robust Method to Produce Pure Drug Nanoparticles With Narrow Size-Dis-tribution, J Pharm Sci 2016, 105(8), 2293-7; WO 2016/055696; and WO 2021/152204.

The properties of the known formulations of Enzalutamide are not satisfactory in every respect. There is a demand for pharmaceutical formulations and dosage forms that contain Enzalutamide and that are advantageous over the prior art, e.g. with respect to the drug load (wt.-%), dosing strength (mg), loading dose, size of dosage form, patient compliance, patient safety due to dosing errors and/or ease of manufacture.

It is therefore an object of the invention to provide pharmaceutical formulations and dosage forms that contain Enzalutamide and that are advantageous over the prior art. In one aspect, the present invention aims at providing pharmaceutical formulations and dosage forms providing immediate release of Enzalutamide. In another aspect, the present invention aims at providing pharmaceutical formulations and oral dosage forms containing a comparatively high dose, preferably 160 mg or more of Enzalutamide per dosage form. It would be desirable to provide a 160 mg tablet that can be swallowed, i.e. that has a total weight of not more than about 1000 mg, and thus a drug load of at least about 16 wt.-%. In still another aspect, the present invention aims at providing pharmaceutical formulations and oral dosage forms that contain Enzalutamide and that show high bioavailability, preferably upon oral administration. In yet another aspect, the present invention aims at providing pharmaceutical formulations and oral dosage forms that can be easily manufactured and are stable.

These objects have been achieved by the subject-matter of the patent claims.

Enzalutamide nanoparticles can be obtained from solution of Enzalutamide in supercritical CO₂ by means of CESS™ technology. It has been found that these nanoparticles contain Enzalutamide in amorphous form (amorphous nanoparticles).

Further, it has been surprisingly found that when nanoparticles containing Enzalutamide in amorphous form (amorphous nanoparticles) are suspended in aqueous solution of suitable polymers and/or copolymers such as copovidone (PVP/VA), crystallization of Enzalutamide is induced; thus obtained nanoparticles contain Enzalutamide in crystalline form (crystalline nanoparticles). This is unusual, because conventional polymeric excipients are rather expected to stabilize an amorphous form, but not to induce or promote crystallization thereof. Aqueous solutions of other polymers such as HPMC or HPMC-AS do not induce crystallization of Enzalutamide.

Still further, it has been surprisingly found that by choosing appropriate conditions crystallization can be controlled and undesirable growth of the crystalline nanoparticles can be prevented. When amorphous nanosized Enzalutamide is crystallized by contacting it with an aqueous solution comprising one or more polymers and/or copolymers to form a suspension or slurry, uncontrolled crystal growth can be avoided or at least alleviated. Thus, the size of the nanoparticles containing Enzalutamide in amorphous form (amorphous nanoparticles) is not significantly increased by inducing crystallization under appropriate conditions. The crystallization as well as the crystal growth is controlled by suitable polymers and/or copolymers in solution, preferably aqueous solution.

Furthermore, it has been surprisingly found that depending upon size, concentration and excipients, nanoparticles comprising Enzalutamide in crystalline form (crystalline nanoparticles) can be prepared that completely or nearly completely disperse from suspension into gastric fluid thereby indicating that such crystalline nanoparticles will likely provide good bioavailability of Enzalutamide when administered in vivo.

The present invention achieves high bioavailability of Enzalutamide by providing it in nanoparticulate form, where the nanoparticles of Enzalutamide in crystalline form do not tend to increase their small size upon storage. The nanoparticles are contained in a composition which additionally comprises one or more physiologically acceptable polymers and/or copolymers. The content of Enzalutamide in the composition can be considerably high. The content of the one or more physiologically acceptable polymers and/or copolymers does not need to exceed the content of Enzalutamide by orders of magnitude in order to achieve the beneficial effects. In consequence, the composition is useful as an intermediate for the preparation of pharmaceutical dosage forms that on the one hand have a high drug load (wt.-%), dosing strength (mg), and/or loading dose, and that on the other hand have a moderate size, thereby facilitating manufacture and improving patient compliance as well as safety.

A first aspect of the invention relates to a composition comprising or essentially consisting of
nanoparticles comprising or essentially consisting of Enzalutamide in crystalline form;
one or more physiologically acceptable polymers and/or copolymers; and
optionally, one or more physiologically acceptable surfactants;
wherein the nanoparticles have a z-average particle size of at most 800 nm; and
wherein the relative weight ratio of the total amount of Enzalutamide to the total amount of the one or more physiologically acceptable polymers and/or copolymers is within the range of from 5.0:1.0 to 1.0:5.0.

For the purpose of the specification, it is distinguished between nanoparticles comprising or essentially consisting of Enzalutamide in crystalline form (also referred to as “crystalline nanoparticles”), and nanoparticles comprising or essentially consisting of Enzalutamide in amorphous form (also referred to as “amorphous nanoparticles”).

Not all constituents of the composition need to be present in crystalline form. In particular, the one or more physiologically acceptable polymers and/or copolymers as well as the optionally present one or more physiologically acceptable surfactants will typically not be present in crystalline form. In preferred embodiments, the one or more physiologically acceptable polymers and/or copolymers as well as the optionally present one or more physiologically acceptable surfactants are even not present in solid form but are dissolved in a liquid phase of a slurry or a suspension, which besides said liquid phase contains the crystalline nanoparticles as a solid phase.

The amorphous nanoparticles are preferably used as starting material for the preparation of the crystalline nanoparticles. The crystalline nanoparticles as well as the amorphous nanoparticles preferably essentially consist of Enzalutamide.

Unless expressly stated otherwise, all percentages are weight percent.

Unless expressly stated otherwise, “essentially consisting of” means to an extent of at least 98%, preferably at least 99%, more preferably about 100%.

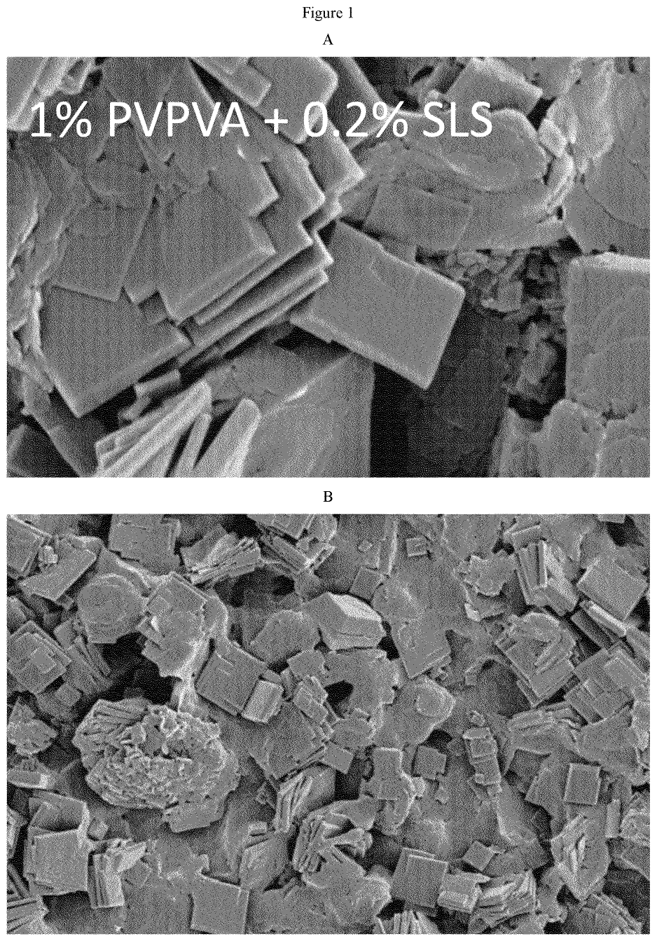

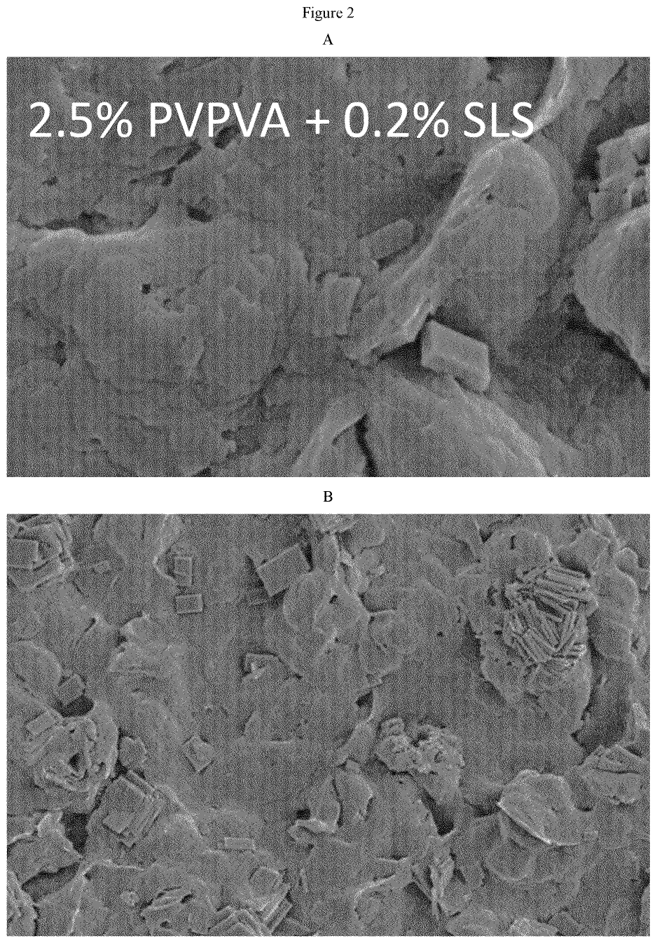

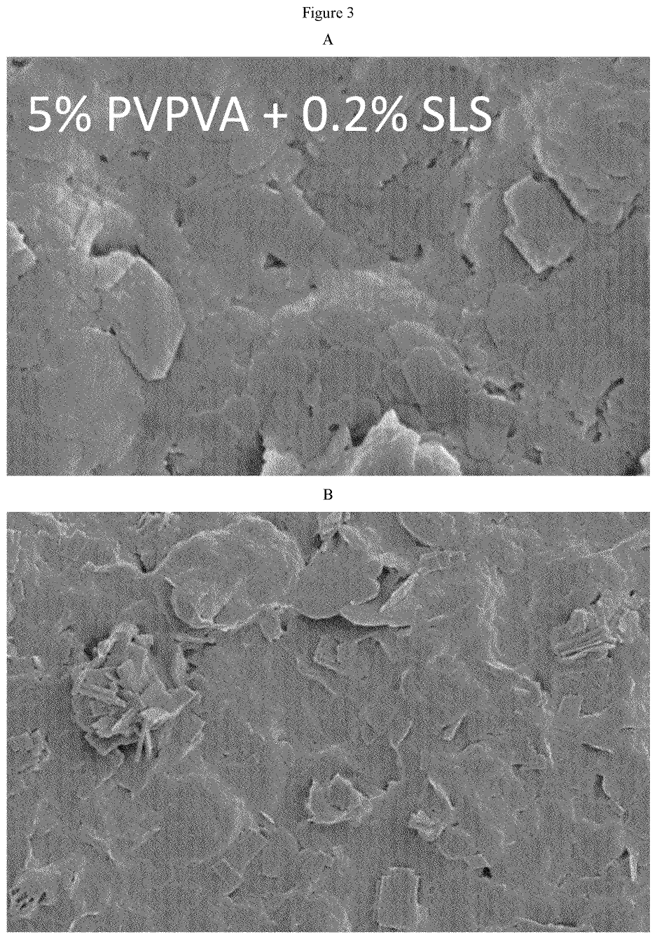

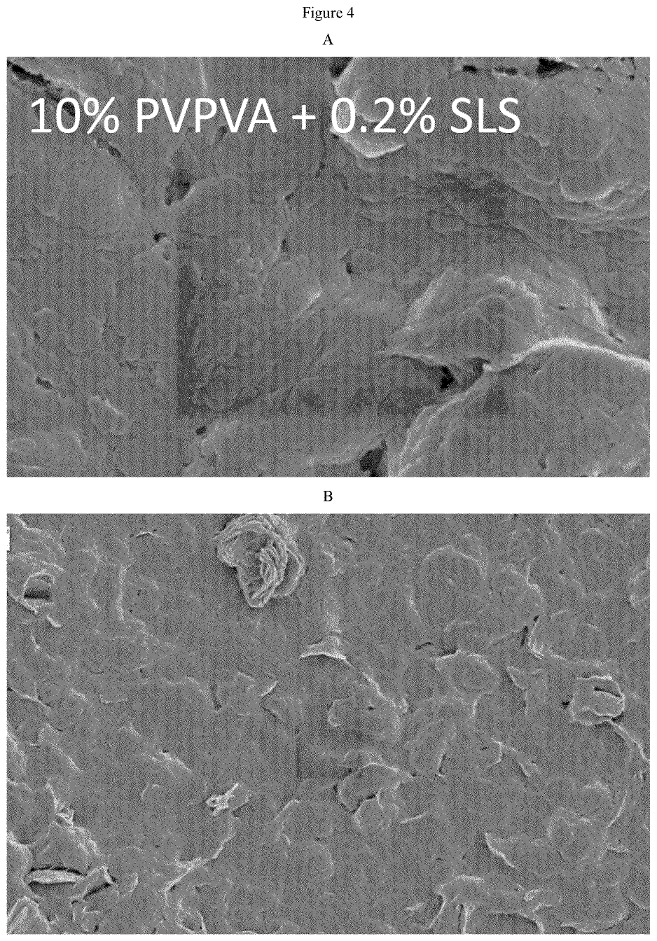

FIGS. 1 to 4 show electron micrographs of crystalline nanoparticles of Enzalutamide that were obtained by stirring amorphous nanoparticles of Enzalutamide in aqueous solutions of PVP-VA at concentrations of 1 wt.-%, 2.5 wt.-%, 5 wt.-% and 10 wt.-% (A: at 25,000 fold magnification, B: at 5,000 fold magnification).

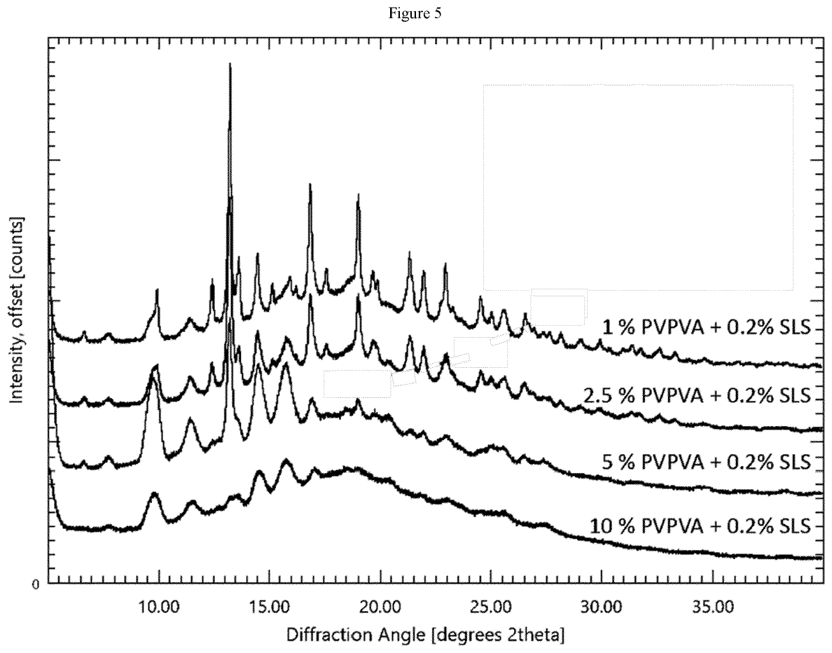

FIG. 5 shows XRPD spectra of crystalline nanoparticles of Enzalutamide obtained from aqueous solutions of PVP-VA at concentrations of 1 wt.-%, 2.5 wt.-%, 5 wt.-% and 10 wt.-%.

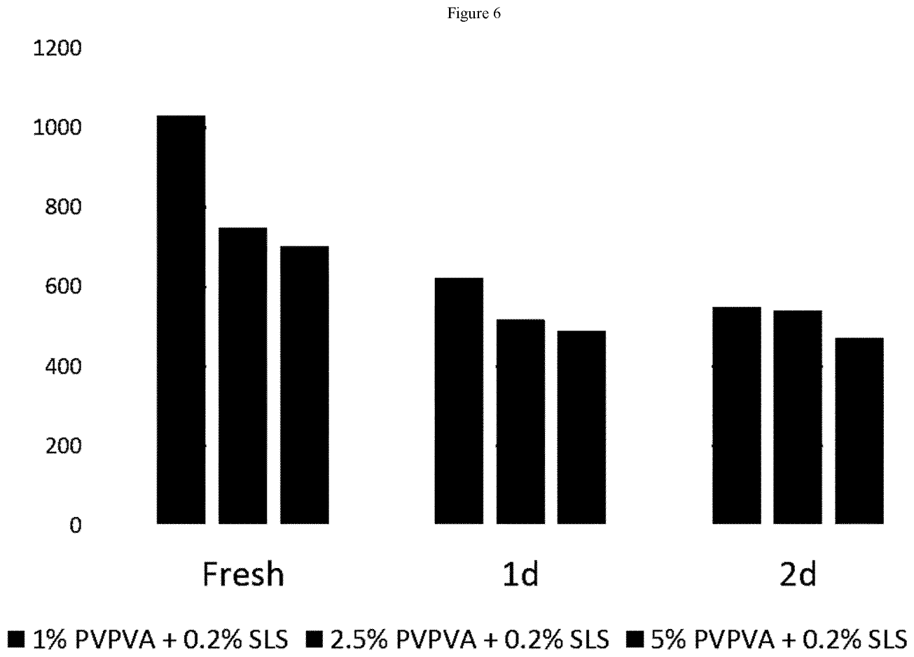

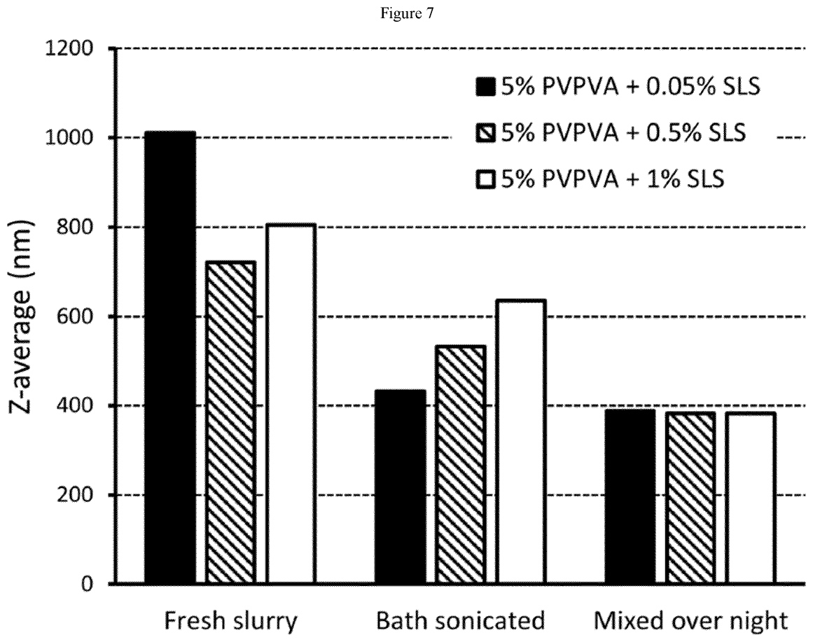

FIGS. 6 and 7 show z-average particle sizes of crystalline nanoparticles obtained after mixing with aqueous solutions of PVP-VA under various conditions.

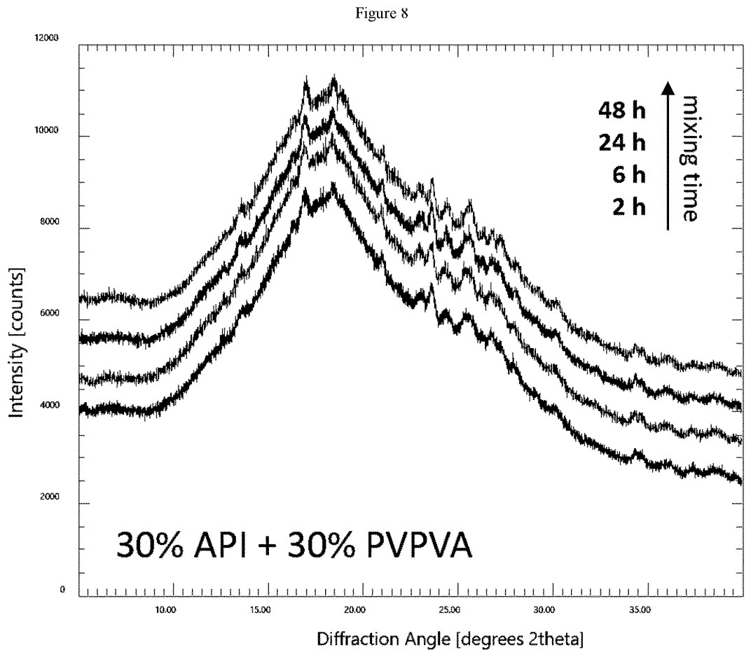

FIG. 8 shows XRPD intensity counts of crystalline nanoparticles of Enzalutamide obtained from suspensions (slurries) in aqueous solutions of PVA-VA at concentrations of 30 wt.-% Enzalutamide and 30 wt.-% PVP-VA at different mixing times.

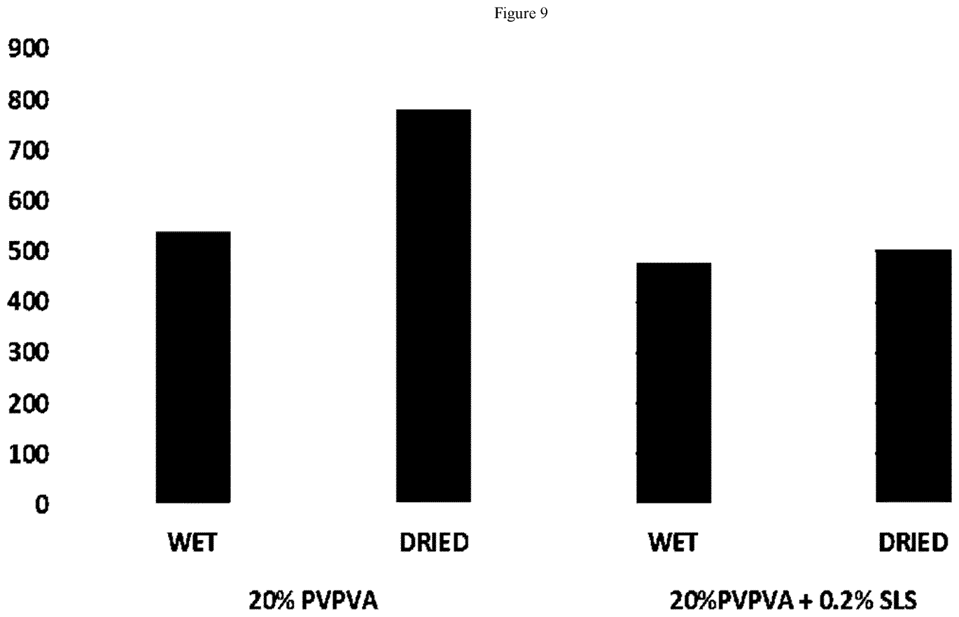

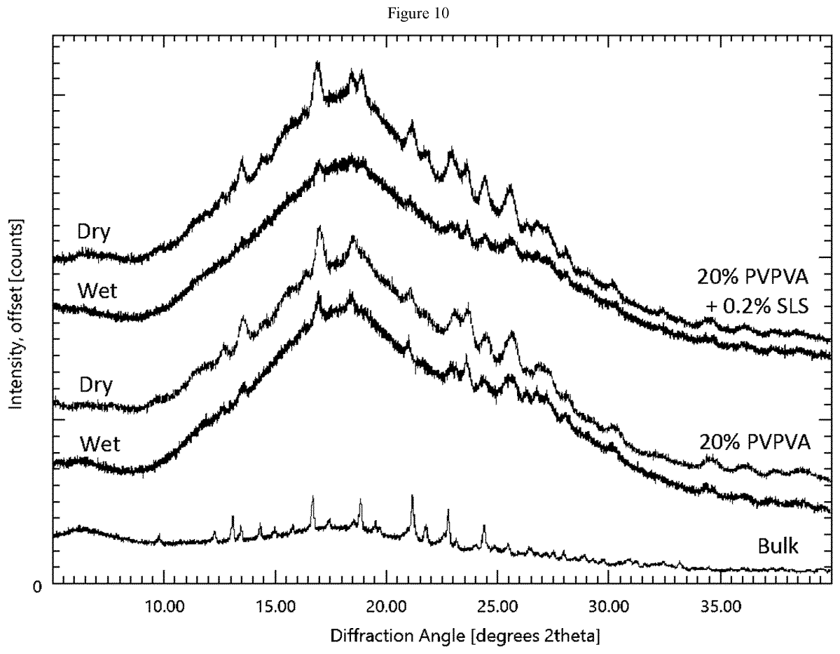

FIG. 9 shows z-average particle sizes in nm, FIG. 10 corresponding XRPD intensity counts of wet and dry crystalline nanoparticles, in the absence and in the presence of SLS.

The composition according to the invention comprises nanoparticles comprising or essentially consisting of Enzalutamide. The nanoparticles according to the invention comprise or essentially consist of Enzalutamide in crystalline form (crystalline nanoparticles).

Enzalutamide is a nonsteroidal antiandrogen (NSAA) medication which is used in the treatment of prostate cancer. It is indicated for use in conjunction with castration in the treatment of metastatic castration-resistant prostate cancer (mCRPC) and nonmetastatic castration-resistant prostate cancer. Enzalutamide is an antiandrogen, and acts as an antagonist of the androgen receptor. It prevents the effects of androgens in the prostate gland.

Enzalutamide (CAS 915087-33-1) has the following chemical structure:

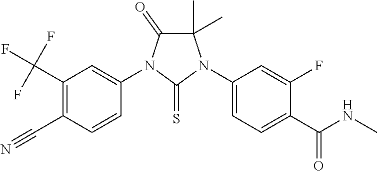 [SMILES: CNC(=O)c1ccc(N2C(=S)N(c3ccc(C#N)c(C(F)(F)F)c3)C(=O)C2(C)C)cc1F]

Enzalutamide is a white-to-off white solid that is insoluble in water. One crystalline form and four solvates have been observed so far. For the purpose of the specification, unless expressly stated otherwise, the term “Enzalutamide” refers to Enzalutamide, its non-salt form, physiologically acceptable salts, co-crystals, polymorphs and/or solvates thereof.

Preferably, the crystalline nanoparticles according to the invention contain Enzalutamide in its non-salt form.

Unless expressly stated otherwise, all dosages and weight percent used herein are based upon the equivalent weight relative to the non-salt form and non-solvate form and non co-crystal form of Enzalutamide, i.e. the additional weight of the salt moiety or solvent moiety or co-crystal moiety is not taken into account for the quantification.

While it is principally contemplated that the composition according to the invention may contain other pharmacologically active ingredients besides the Enzalutamide, Enzalutamide is preferably the sole pharmacologically active ingredient that is contained in the composition. In this context, pharmacologically active ingredients are other substances that are useful in treating the same or related disorders and diseases and conditions as Enzalutamide. Thus, compounds having a physiological but no pharmacological effect such as sodium chloride, vitamins and the like, are not to be regarded as pharmacologically active ingredients in the above meaning.

Preferably, the crystalline nanoparticles within the composition according to the invention are solid.

Preferably, the crystalline nanoparticles according to the invention and the Enzalutamide contained therein are not conjugated to antigens, e.g. for the purposes of drug targeting. In particular, the crystalline nanoparticles according to the invention are not encapsulated in a prostate specific membrane antigen (PSMA), i.e. are not coated with PSMA.

Preferably, the content of Enzalutamide, relative to the total weight of the nanoparticles, is at least 90.0 wt.-%, preferably at least 92.5 wt.-%, more preferably at least 95 wt.-%, still more preferably at least 96 wt.-%, yet more preferably at least 97 wt.-%, even more preferably at least 98 wt.-%, most preferably at least 99.0 wt.-%, and in particular at least 99.5 wt.-%.

Preferably, the content of Enzalutamide, relative to the total dry solids content of the composition, is at least 10 wt.-%, preferably at least 15 wt.-%, more preferably at least 20 wt.-%, still more preferably at least 25 wt.-%, yet more preferably at least 30 wt.-%, even more preferably at least 35 wt.-%, most preferably at least 40 wt.-%, and in particular at least 45 wt.-%.

Preferably, the content of Enzalutamide, relative to the total dry solids content of the composition, is at most 90 wt.-%, preferably at most 85 wt.-%, more preferably at most 80 wt.-%, still more preferably at most 75 wt.-%, yet more preferably at most 70 wt.-%, even more preferably at most 65 wt.-%, most preferably at most 60 wt.-%, and in particular at most 55 wt.-%.

Preferably, the content of Enzalutamide, relative to the total weight of the composition, is at least 7.5 wt.-%, preferably at least 10 wt.-%, more preferably at least 12.5 wt.-%, still more preferably at least 15 wt.-%, yet more preferably at least 17.5 wt.-%, even more preferably at least 20 wt.-%, most preferably at least 22.5 wt.-%, and in particular at least 25 wt.-%.

Preferably, the content of Enzalutamide, relative to the total weight of the composition, is at most 60 wt.-%, preferably at most 55 wt.-%, more preferably at most 50 wt.-%, still more preferably at most 45 wt.-%, yet more preferably at most 40 wt.-%, even more preferably at most 35 wt.-%, most preferably at most 30 wt.-%, and in particular at most 25 wt.-%.

In the crystalline nanoparticles according to the invention, Enzalutamide is present in crystalline form. Thus, it is contemplated that the crystalline nanoparticles comprise crystalline Enzalutamide and optionally, also non-crystalline (i.e. amorphous) Enzalutamide.

In preferred embodiments, the crystalline nanoparticles according to the invention are nanoflakes, i.e. uneven particles with one dimension substantially smaller than the other two with at least one nanometric dimension, characterized by a plate-like form or structure.

The crystalline nanoparticles according to the invention have a z-average particle size of at most 800 nm.

In preferred embodiments, particle size and particle size distribution of the crystalline nanoparticles according to the invention are expressed in terms of the z-average particle size Dz.

Preferably, the crystalline nanoparticles according to the invention have a z-average particle size of at most 750 nm, preferably at most 700 nm, more preferably at most 650 nm, still more preferably at most 600 nm, yet more preferably at most 550 nm, even more preferably at most 500 nm, most preferably at most 450 nm, and in particular at most 400 nm.

Preferably, the crystalline nanoparticles according to the invention have a z-average particle size Dz of at least 10 nm, preferably at least 20 nm, more preferably at least 30 nm, still more preferably at least 40 nm, yet more preferably at least 50 nm, even more preferably at least 60 nm, most preferably at least 70 nm, and in particular at least 80 nm.

Preferably, unless expressly stated otherwise, the z-average particle size (Dz) and the particle size distribution of the crystalline nanoparticles according to the invention are determined in accordance with ISO 22412:2008 Particle Size Analysis-Dynamic Light Scattering. The z-average particle size Dz is the intensity based harmonic mean (also known as the “cumulants mean”).

Dynamic light scattering (DLS) measurements are preferably performed with a Malvern Zetasizer Nano device, e.g. Zetasizer Nano ZS. The composition according to the invention (dry powder of slurry/suspension) is redispersed into water or into 0.1% HPMC (aq.), stirred and measured after the sample is completely dispersed. Backscattering measurement setup is preferably used, and CUMULANTS-algorithm is preferably used to obtain z-average particle size (diameter) and polydispersity index (PI). For details, reference is made e.g. to the user manual Zetasizer nano series, NANO485 Issue 1.1 Apr. 2013.

In other preferred embodiments, particle size and particle size distribution of the crystalline nanoparticles according to the invention are expressed in terms of the Dᵥ50 and/or Dᵥ90.

Preferably, the crystalline nanoparticles according to the invention have a particle size distribution characterized by a Dᵥ90 value equal to or less than 900 nm, i.e., the crystalline nanoparticles consist of a multitude of particles, wherein 90% of the volume fraction has a diameter equal to or less than 900 nm. The particle size may be between 10 nm and 900 nm, for example between 10 nm and 200 nm, between 200 nm and 500 nm, or between 500 nm and 900 nm. The size distribution can be tuned as desired.

Preferably, the crystalline nanoparticles according to the invention have a particle size distribution characterized by a Dᵥ90 value of at most 600 nm, preferably at most 550 nm, more preferably at most 500 nm, still more preferably at most 450 nm, yet more preferably at most 400 nm, even more preferably at most 350 nm, most preferably at most 300 nm, and in particular at most 250 nm.

Preferably, the crystalline nanoparticles according to the invention have a particle size distribution characterized by a Dᵥ50 value of at most 600 nm, preferably at most 550 nm, more preferably at most 500 nm, still more preferably at most 450 nm, yet more preferably at most 400 nm, even more preferably at most 350 nm, most preferably at most 300 nm, and in particular at most 250 nm.

Preferably, unless expressly stated otherwise, Dᵥ50 and Dᵥ90 are determined by laser light diffraction, which provides distribution by volume (Dᵥ), preferably in accordance with Ph. Eur., 2.9.31 “Particle Size Analysis By Laser Light Diffraction”. Samples of known particle size distribution are commercially available and can be used for calibration.

In preferred embodiments, the particle size is analyzed by Polarization Intensity Differential Scattering (PIDS) technology, which is based on Mie theory of light scattering using different wavelength and measuring the difference between vertically and horizontally polarized signals. A suitable device is e.g. LS 13 320 XR Particle Size Analyzer, Beckman Coulter Inc.

The width of the particle size distribution in suspension is characterized by the “polydispersity” or “PDI” of the crystalline nanoparticles, which is defined as the relative variance in the correlation decay rate distribution, as is known by one skilled in the art. The polydispersity index (PDI) can also be calculated from the cumulants analysis of the DLS measured intensity autocorrelation function as defined in ISO22412:2008. Preferably, the polydispersity of the crystalline nanoparticles according to the invention is less than 0.6, or less than 0.5, or less than 0.4, or less than 0.3, or less than 0.2, or less than 0.1.

The composition according to the invention comprises one or more physiologically acceptable polymers and/or copolymers. For the purpose of the specification, a copolymer is derived from at least two different monomers (comonomers). Preferably, the physiologically acceptable copolymer is a bipolymer, i.e. derived from two different monomers (comonomers).

It has been surprisingly found that in order to induce and control crystallization of Enzalutamide, the one or more physiologically acceptable polymers and/or copolymers are preferably soluble in an antisolvent for Enzalutamide, preferably water.

Preferably, the one or more physiologically acceptable polymers and/or copolymers can be synthetic polymers or biopolymers such as proteins. Exemplary polymers are polyvinylpyrrolidone/vinyl acetate (PVPVA), polyvinyl acetate (PVA), polyvinyl pyrrolidone (PVP), polyacrylic acid (PAA), polyethylene glycol (PEG), poloxamers, polyvinyl caprolactam (PVCL), poly(N-vinyl caprolactam)poly(vinyl acetate)-poly(ethylene glycol) (Soluplus®), and any co-polymer or mixture of the foregoing. Also, proteins such as wheat proteins can be used. A preferable copolymer is PVPVA.

Preferably, the one or more physiologically acceptable polymers and/or copolymers have a solubility in water according to Ph. Eur. (between 15 and 25° C.) of at least “sparingly soluble” (30 to 100 mL of water per g), preferably at least “soluble” (10 to 30 mL of water per g), more preferably at least “freely soluble” (1 to 10 mL of water per g).

Further, it has been surprisingly found that in order to induce and control crystallization of Enzalutamide, the one or more physiologically acceptable polymers and/or copolymers should be composed of repetition units having different hydrophilicity and hydrophobicity, respectively.

Preferably, the one or more physiologically acceptable polymers and/or copolymers have sufficient hydrophilicity to be water soluble and sufficient lipophilicity to interact with Enzalutamide.

Preferably, the one or more physiologically acceptable polymers and/or copolymers comprise or essentially consist of a copolymer that is derived from a first monomer and a second monomer, wherein the first monomer is more hydrophilic than the second monomer. Preferably, the first monomer in its neat state has a dipole moment that is relatively at least 0.1 Debye, preferably at least 0.2 Debye, more preferably at least 0.3 Debye, still more preferably at least 0.4 Debye greater than the dipole moment of the second monomer in its neat state.

In preferred embodiments, the one or more physiologically acceptable polymers and/or co-polymers comprise or essentially consist of a vinylpyrrolidone vinylacetate copolymer (copovidone). Such polymers are commercially available, e.g. as Kollidon® VA64, or Copovidone K25-31.

Preferably, the vinylpyrrolidone vinylacetate copolymer is a copolymer of 1-vinyl-2-pyrrolidone (1-ethenylpyrrolidin-2-one) and vinylacetate (ethenyl acetate) in the mass proportion of about 3:2.

Preferably, the vinylpyrrolidone vinylacetate copolymer contains not less than 35.0 wt.-% and not more than 42.0 wt.-% of vinylacetate (ethenyl acetate), calculated on the dried basis.

Preferably, the total content of the one or more physiologically acceptable polymers and/or co-polymers, relative to the total dry solids content of the composition, is at least 10 wt.-%, preferably at least 15 wt.-%, more preferably at least 20 wt.-%, still more preferably at least 25 wt.-%, yet more preferably at least 30 wt.-%, even more preferably at least 35 wt.-%, most preferably at least 40 wt.-%, and in particular at least 45 wt.-%.

Preferably, the total content of the one or more physiologically acceptable polymers and/or co-polymers, relative to the total dry solids content of the composition, is at most 90 wt.-%, preferably at most 85 wt.-%, more preferably at most 80 wt.-%, still more preferably at most 75 wt.-%, yet more preferably at most 70 wt.-%, even more preferably at most 65 wt.-%, most preferably at most 60 wt.-%, and in particular at most 55 wt.-%.

Preferably, the total content of the one or more physiologically acceptable polymers and/or co-polymers, relative to the weight of the composition, is at least 7.5 wt.-%, preferably at least 10 wt.-%, more preferably at least 12.5 wt.-%, still more preferably at least 15 wt.-%, yet more preferably at least 17.5 wt.-%, even more preferably at least 20 wt.-%, most preferably at least 22.5 wt.-%, and in particular at least 25 wt.-%.

Preferably, the total content of the one or more physiologically acceptable polymers and/or co-polymers, relative to the total weight of the composition, is at most 60 wt.-%, preferably at most 55 wt.-%, more preferably at most 50 wt.-%, still more preferably at most 45 wt.-%, yet more preferably at most 40 wt.-%, even more preferably at most 35 wt.-%, most preferably at most 30 wt.-%, and in particular at most 25 wt.-%.

The relative weight ratio of the total amount of Enzalutamide to the total amount of the one or more physiologically acceptable polymers and/or copolymers is within the range of from 5.0:1.0 to 1.0:5.0.

In preferred embodiments, the relative weight ratio of the total amount of Enzalutamide to the total amount of the one or more physiologically acceptable polymers and/or copolymers is within the range of from 4.5:1.0 to 1.0:4.5, preferably from 4.0:1.0 to 1.0:4.0, more preferably from 3.5:1.0 to 1.0:3.5, still more preferably from 3.0:1.0 to 1.0:3.0, yet more preferably from 2.5:1.0 to 1.0:2.5, even more preferably from 2.0:1.0 to 1.0:2.0, most preferably from 1.5:1.0 to 1.0:1.5, and in particular from 1.3:1.0 to 1.0:1.3.

In preferred embodiments, the composition according to the invention is a suspension or a slurry comprising or essentially consisting of (i) a solid phase, preferably comprising or essentially consisting of the nanoparticles comprising or essentially consisting of Enzalutamide in crystalline form; and (ii) a liquid phase, preferably comprising or essentially consisting of a liquid (e.g. solvent, preferably water); the one or more physiologically acceptable polymers and/or copolymers; and optionally, the one or more physiologically acceptable surfactants; wherein the relative weight ratio of the total amount of Enzalutamide to the total amount of the one or more physiologically acceptable polymers and/or co-polymers is within the range of from 2.0:1.0 to 2.0:3.0, wherein the amount of Enzalutamide is calculated as mg/mL of the slurry or suspension, and the amount of polymer and/or copolymer is calculated as wt.-% of the slurry or suspension.

The composition according to the invention optionally comprises one or more physiologically acceptable surfactants.

In preferred embodiments, the composition according to the invention does not comprise any surfactant. Preferably, the composition is free of sodium lauryl sulfate; more preferably free of any anionic surfactant; still more preferably free of any physiologically acceptable surfactants.

In other preferred embodiments, the composition according to the invention contains one or more physiologically acceptable surfactants. The surfactants enhance the wetting efficiency.

The properties of surfactants may be described by their hydrophilic-lipophilic-balance (HLB). Preferably, the one or more physiologically acceptable surfactants comprise or essentially consist of a surfactant having a HLB value of at least 10, preferably at least 15, more preferably at least 20, still more preferably at least 25, yet more preferably at least 30, even more preferably at least 32, most preferably at least 34, and in particular at least 36.

The properties of surfactants may also be described by their charge.

In a preferred embodiment, the one or more physiologically acceptable surfactants comprise or essentially consist of a nonionic surfactant.

In a preferred embodiment, the one or more physiologically acceptable surfactants comprise or essentially consist of an anionic surfactant.

In a preferred embodiment, the one or more physiologically acceptable surfactants comprise or essentially consist of a cationic surfactant.

In a preferred embodiment, the one or more physiologically acceptable surfactants comprise or essentially consist of an amphoteric surfactant.

Preferably, the one or more physiologically acceptable surfactants comprise or essentially consist of a surfactant selected from the group consisting of
(i) alkyl sulfate salts; preferably selected from sodium lauryl sulfate (sodium dodecyl sulfate), sodium cetyl sulfate, sodium cetylstearyl sulfate, sodium stearyl sulfate, sodium dioctylsulfosuccinate (docusate sodium); and the corresponding potassium or calcium salts thereof;
(ii) fatty acid salts; preferably selected from stearic acid salts, oleic acid salts;
(iii) salts of cholic acid; preferably selected from sodium deoxycholate, sodium glycocholate, sodium taurocholate and the corresponding potassium or ammonium salts.

Preferably, the one or more physiologically acceptable surfactants comprise or essentially consist of an alkyl sulfate salt; preferably of the general formula CₙH₂ₙ₊₁O—SO₃⁻M⁺, wherein n is an integer of from 8 to 30, preferably 10 to 24, more preferably 12 to 18; and M is selected from Lit, Na+, K+, NH₄⁺, 1/2 Mg²⁺ and 1/2 Ca²⁺.

In particularly preferred embodiments, the one or more physiologically acceptable surfactants comprise or essentially consist of sodium lauryl sulfate.

Preferably, the one or more physiologically acceptable surfactants comprise or essentially consist of a surfactant selected from the group consisting of
(a) straight or branched chain fatty alcohols; preferably selected from cetyl alcohol, cetostearyl alcohol, stearyl alcohol, oleyl alcohol, octyldodecanol, or 2-hexyldecane-1-ol;
(b) sterols; preferably cholesterol;
(c) lanolin alcohols;
(d) partial fatty acid esters of multivalent alcohols, e.g. glycerol fatty acid monoesters or glycerol fatty acid diesters; preferably selected from glycerol behenate, glycerol dibehenate, glycerol distearate, glycerol monocaprylate, glycerol monolinoleate, glycerol mono oleate, glycerol monostearate, ethylene glycol monopalmitostearate, ethylene glycol stearate, diethylene glycol palmitostearate, diethylene glycol stearate, propylene glycol dicaprylocaprate, propylene glycol dilaurate, propylene glycol monocaprylate, propylene glycol monolaurate, propylene glycol monopalmitostearate, propylene glycol monostearate, pentaerythritol monostearate, superglycerinated fully hydrogenated rapeseed oil;
(e) partial fatty acid esters of sorbitan; preferably selected from sorbitan monolaurate, sorbitan monopalmitate, sorbitan monostearate, sorbitan tristearate, sorbitan monooleate, sorbitan sesquioleate, sorbitan trioleate;
(f) partial fatty acid esters of polyoxyethylene sorbitan, (polyoxyethylene-sorbitan-fatty acid esters), e.g. fatty acid monoesters of polyoxyethylene sorbitan, a fatty acid diesters of polyoxyethylene sorbitan, or a fatty acid triesters of polyoxyethylene sorbitan; such as mono- and tri-lauryl, palmityl, stearyl and oleyl esters; preferably selected from polyoxy-ethylene(20)sorbitan monolaurate, polyoxyethylene(4)sorbitan monolaurate, polyoxy-ethylene(20)sorbitan monopalmitate, polyoxyethylene(20)sorbitan monostearate, polyoxy-ethylene(20)sorbitan tristearate, polyoxyethylene(20)sorbitan monooleate, polyoxyethylene(5)sorbitan monooleate, polyoxyethylene(20)sorbitan trioleate;
(g) polyoxyethyleneglycerole fatty acid esters, e.g. mixtures of mono-, di- and triesters of glycerol and di- and monoesters of macrogols having molecular weights within the range of from 200 to 4000 g/mol; preferably selected from macrogolglycerolcaprylocaprate, macrogolglycerollaurate, macrogolglycerolococoate, macrogolglycerollinoleate, macrogol-20-glycerolmonostearate, macrogol-6-glycerolcaprylocaprate, macrogolglycerololeate; macrogolglycerolstearate, macrogolglycerolhydroxystearate, macrogolglycerolrizinoleate;
(h) polyoxyethylene fatty acid esters; preferably selected from macrogololeate, macrogolstearate, macrogol-15-hydroxystearate, polyoxyethylene esters of 12-hydroxystearic acid;
(i) fatty alcohol ethers of polyoxyethylene; preferably selected from polyoxyethylene lauryl ether, polyoxyethylene cetyl ether, polyoxyethylene stearyl ether, polyoxyethylene oleyl ether, polyoxyethylene cetostearyl ether, lauromacrogol 400, macrogol oleyl ether, macrogol stearyl ether;
(j) reaction products of a natural or hydrogenated castor oil and ethylene oxide such as those commercialized as Cremophor®;
(k) polyoxypropylene-polyoxyethylene blockcopolymers (poloxamers); preferably according to the following general formula

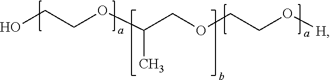

wherein a is an integer independently within the range of from 2 to 130, preferably from 90 to 110; and wherein b is an integer within the range of from 15 to 67, preferably from 46 to 66;
(l) polyglycolyzed glycerides; preferably selected from those commercialized as Gelucire®, Labrasol®;
(m) fatty acid esters of sucrose; preferably selected from sucrose distearate, sucrose dioleate, sucrose dipalmitate, sucrose monostearate, sucrose monopalmitate, sucrose monooleate, sucrose monomyristate, sucrose mololaurate;
(n) fatty acid esters of polyglycerol; preferably selected from polyglycerol oleate polyglycerol dioleate, polyglycerol poly-12-hydroxystearate, triglycerol diisostearate; and
(o) polyoxyethylene esters of D-α-tocopheryl succinate; preferably D-α-tocopherol polyethylene glycol 1000 succinate.

Preferably, the one or more physiologically acceptable surfactants comprise or essentially consist of a surfactant selected from the group consisting of sodium lauryl sulfate (SLS) Tween 80, Tween 20, dioctyl sulfosuccinate sodium salt (DOSS), and tocofersolan (TPGS).

Preferably, the total content of the one or more physiologically acceptable surfactants, relative to the total weight of the composition, is at most 7.0 wt.-%, preferably at most 6.0 wt.-%, still more preferably at most 5.0 wt.-%, yet more preferably at most 4.0 wt.-%, even more preferably at most 3.0 wt.-%, most preferably at most 2.0 wt.-%, and in particular at most 1.0 wt.-%.

Preferably, the composition is a suspension or a slurry comprising or essentially consisting of (i) a solid phase comprising or essentially consisting of the nanoparticles and (ii) a liquid phase, wherein the total content of the one or more surfactants in the liquid phase is within the range from 0.0025 to 1.5 wt.-%.

In particularly preferred embodiments, the composition according to the invention comprises or essentially consists of
nanoparticles comprising or essentially consisting of Enzalutamide in crystalline form;
a vinylpyrrolidone vinylacetate copolymer; and
optionally, sodium lauryl sulfate;
wherein the nanoparticles have a z-average particle size of at most 700 nm; preferably at most 600 nm; more preferably at most 500 nm; and
wherein the relative weight ratio of the total amount of Enzalutamide to the vinylpyrrolidone vinylacetate copolymer is within the range of from 4.0:1.0 to 1.0:4.0; preferably from 2.5:1.0 to 1.0:2.5

In preferred embodiments, the composition according to the invention is a solid.

Under these circumstances, the composition according to the invention preferably essentially consists of
the nanoparticles comprising or essentially consisting of Enzalutamide in crystalline form;
the one or more physiologically acceptable polymers and/or copolymers; and
optionally, the one or more physiologically acceptable surfactants.

In other preferred embodiments, the composition according to the invention is a suspension or a slurry comprising or essentially consisting of
(i) a solid phase, preferably comprising or essentially consisting of the nanoparticles comprising or essentially consisting of Enzalutamide in crystalline form; and
(ii) a liquid phase, preferably comprising or essentially consisting of
   a liquid (e.g. solvent, preferably water);
   the one or more physiologically acceptable polymers and/or copolymers; and
   optionally, the one or more physiologically acceptable surfactants.

Preferably, the liquid phase is aqueous. Preferably, water is the only liquid constituent of the composition, while it is contemplated that physiologically acceptable surfactants may be liquids in their neat state such that the liquid constituents of the liquid phase may essentially consist of water and such liquid physiologically acceptable surfactant(s).

Preferably, at least a portion, more preferably essentially the total amount of the one or more physiologically acceptable polymers and/or copolymers is dissolved in the liquid phase.

Preferably, at least a portion, more preferably essentially the total amount of the optionally present one or more physiologically acceptable surfactants is dissolved in the liquid phase.

In preferred embodiments, the composition according the invention has a liquid content, preferably water content, of at least 5.0 wt.-%, preferably at least 10 wt.-%, more preferably at least 15 wt.-%, still more preferably at least 20 wt.-%, yet more preferably at least 25 wt.-%, even more preferably at least 30 wt.-%, most preferably at least 25 wt.-%, and in particular at least 40 wt.-%, in each case relative to the total weight of the composition (i.e. suspension or slurry).

In particularly preferred embodiments of the composition according to the invention, relative to the total weight of the composition
the content of Enzalutamide is within the range of 25±20 wt.-%, preferably 25±15 wt.-%, more preferably 25±10 wt.-%, still more preferably 25±5.0 wt.-%;
the total content of the one or more physiologically acceptable polymers and/or copolymers is within the range of 25±20 wt.-%, preferably 25±15 wt.-%, more preferably 25±10 wt.-%, still more preferably 25±5.0 wt.-%; and
the content of water is within the range of 50±40 wt.-%, preferably 50±30 wt.-%, more preferably 50±20 wt.-%, still more preferably 50±10 wt.-%.

Preferably, the composition according to the invention has a viscosity of at least 5,000 mPa·s, preferably at least 7,500 mPa·s, more preferably at least 10,000 mPa's at a temperature within the range of from 15° C. to 40° C.

Preferably, the composition according to the invention has a viscosity of at most 40,000 mPa·s, preferably at most 35,000 mPa·s, more preferably at most 30,000 mPa·s at a temperature within the range of from 15° C. to 40° C.

Preferably, the composition according to the invention contains the nanoparticles at a total concentration, relative to the total weight of the composition (slurry or suspension), of at least 7.5 mg/mL, preferably at least 10 mg/mL, more preferably at least 12.5 mg/mL, still more preferably at least 15 mg/mL, yet more preferably at least 17.5 mg/mL, even more preferably at least 20 mg/mL, most preferably at least 22.5 mg/mL, and in particular at least 25 mg/mL.

Preferably, the composition according to the invention contains the nanoparticles at a total concentration, relative to the total weight of the composition (slurry or suspension), of at least 75 mg/mL, preferably at least 100 mg/mL, more preferably at least 125 mg/mL, still more preferably at least 150 mg/mL, yet more preferably at least 175 mg/mL, even more preferably at least 200 mg/mL, most preferably at least 225 mg/mL, and in particular at least 250 mg/mL.

Preferably, the composition according to the invention contains the nanoparticles at a total concentration, relative to the total weight of the composition (slurry or suspension), of at most 475 mg/mL, preferably at most 450 mg/mL, more preferably at most 425 mg/mL, still more preferably at most 400 mg/mL, yet more preferably at most 375 mg/mL, even more preferably at most 350 mg/mL, most preferably at most 325 mg/mL, and in particular at most 300 mg/mL.

Preferably, the composition according to the invention contains the nanoparticles at a total concentration, relative to the total weight of the composition (slurry or suspension), of at most 47.5 mg/mL, preferably at most 45 mg/mL, more preferably at most 42.5 mg/mL, still more preferably at most 40 mg/mL, yet more preferably at most 37.5 mg/mL, even more preferably at most 35 mg/mL, most preferably at most 32.5 mg/mL, and in particular at most 30 mg/mL.

The synthesis strategy according to the invention involving treatment of amorphous nanoparticles of Enzalutamide with a solution of one or more physiologically acceptable polymers and/or co-polymers in an antisolvent for Enzalutamide, preferably in water, is unique and has unexpected advantages, because
(i) the Enzalutamide in the nanoparticles is converted from its initial amorphous state (amorphous nanoparticles) into the crystalline state (crystalline nanoparticles);
(ii) the small initial size of the amorphous nanoparticles essentially consisting of Enzalutamide molecules (starting material) is not significantly increased by conversion from the initial amorphous state into the crystalline state; and
(iii) the small size of the thus obtained crystalline nanoparticles is stabilized by the presence of the one or more physiologically acceptable polymers and/or copolymers in the composition thus providing satisfactory storage stability of the composition.

Another aspect of the invention relates to a process for converting nanoparticles comprising or essentially consisting of Enzalutamide in amorphous form (amorphous nanoparticles) into nanoparticles comprising Enzalutamide in crystalline form (crystalline nanoparticles).

Another aspect of the invention relates to a process for the preparation of a composition comprising or essentially consisting of nanoparticles comprising or essentially consisting of Enzalutamide in crystalline form (crystalline nanoparticles), one or more physiologically acceptable polymers and/or copolymers, and optionally, one or more physiologically acceptable surfactants, according to the invention as described above.

In either case, the process comprises the steps of
(a) providing nanoparticles comprising or essentially consisting of Enzalutamide in amorphous form;
(b) contacting the nanoparticles provided in step (a) with the one or more physiologically acceptable polymers and/or copolymers in a liquid thereby obtaining a suspension or a slurry;
(c) optionally, mixing the suspension or the slurry;
(d) optionally, drying the suspension or the slurry thereby obtaining a residual composition; and
(e) optionally, crushing the residual composition;
thereby obtaining the nanoparticles comprising or essentially consisting of Enzalutamide in crystalline form (crystalline nanoparticles).

In step (a) of the process according to the invention, nanoparticles are provided that comprise or essentially consist of Enzalutamide in amorphous form (amorphous nanoparticles, starting material). This can be achieved by conventional methods known from the prior art including comminution processes (top-down methods) such as wet nanogrinding using stirred media mills or high-pressure homogenization; or nanoprecipitation (bottom-up methods). Preferably, step (a) involves dissolving Enzalutamide in supercritical CO₂ and providing the nanoparticles that comprise or essentially consist of Enzalutamide in amorphous form (amorphous nanoparticles) by means of CESS™ technology.

Preferably, the amorphous nanoparticles provided in step (a) have a z-average particle size of at most 600 nm, preferably at most 550 nm, more preferably at most 500 nm, still more preferably at most 450 nm, yet more preferably at most 400 nm, even more preferably at most 350 nm, most preferably at most 300 nm, and in particular at most 250 nm.

Preferably, the amorphous nanoparticles provided in step (a) have a z-average particle size of at least 60 nm, preferably at least 80 nm, more preferably at least 100 nm, still more preferably at least 120 nm, yet more preferably at least 140 nm, even more preferably at least 160 nm, most preferably at least 180 nm, and in particular at least 200 nm.

Preferably, the amorphous nanoparticles provided in step (a) have a particle size distribution characterized by a Dᵥ90 value of at most 1000 nm, preferably at most 900 nm, more preferably at most 800 nm, still more preferably at most 700 nm, yet more preferably at most 650 nm, even more preferably at most 600 nm, most preferably at most 550 nm, and in particular at most 500 nm.

Preferably, the amorphous nanoparticles provided in step (a) have a particle size distribution characterized by a Dᵥ50 value of at least 50 nm, preferably at least 100 nm, more preferably at least 150 nm, still more preferably at least 200 nm, yet more preferably at least 250 nm, even more preferably at least 300 nm, most preferably at least 350 nm, and in particular at least 400 nm.

In step (b) of the process according to the invention, the amorphous nanoparticles provided in step (a) are suspended in a liquid comprising the one or more physiologically acceptable polymers and/or copolymers and optionally, the one or more physiologically acceptable surfactants, thereby obtaining a suspension or a slurry.

Typically, the liquid on the one hand and the nanoparticles on the other hand are separate phases of the slurry or suspension, i.e. liquid phase and solid phase, respectively.

Preferably, in step (b) the liquid is a solution of the one or more physiologically acceptable polymers and/or copolymers and optionally, the one or more physiologically acceptable surfactants.

Preferably, in step (b) the liquid is aqueous; preferably wherein water is the only liquid constituent.

Preferably, the amorphous nanoparticles provided in step (a) (amorphous nanosized Enzalutamide), and the liquid, preferably aqueous solution, comprising the one or more polymers and/or co-polymers are contacted with one another to form a slurry or suspension. The content of the amorphous nanoparticles provided in step (a) (amorphous nanosized Enzalutamide), in the slurry or suspension is higher than the solubility of the amorphous nanoparticles provided in step (a) (amorphous nanosized Enzalutamide) in the liquid, preferably aqueous solution, thereby producing the suspension or the slurry.

Preferably, the content of the amorphous nanosized Enzalutamide in the slurry or suspension is at least 10 times higher, more preferably at least 50 times, still more preferably at least 100 times higher, even more preferably at least 500 times higher than its solubility in the liquid, preferably aqueous solution, comprising the one or more polymers and/or copolymers to form the suspension or the slurry. Accordingly, the amount of Enzalutamide amorphous nanoparticles provided in step (a) that is needed depends on its solubility. The solubility can be measured by any method known in the art. The kinetic solubility of Enzalutamide at 37° C. in Fasted State Simulated Intestinal Fluid (FaSSIF) is about 74 mg/mL and in Simulated Gastric Fluid (SGF) is about 50 mg/mL. A decent slurry can thus be produced when the slurry or suspension comprises e.g. 5 mg of the amorphous nanoparticles provided in step (a) in 1 mL of the slurry or suspension.

Preferably, in step (b) the liquid, preferably aqueous solution, contains the one or more physiologically acceptable polymers and/or copolymers at a concentration within the range of from 0.2 to 40 wt.-% by weight, preferably from 1 to 10 wt.-%, more preferably from 1 to 5.0 wt.-%, relative to the total weight of the liquid. Too high polymer content may lead to slow crystal formation.

Preferably, in step (b) the liquid contains the one or more physiologically acceptable polymers and/or copolymers at a total concentration, relative to the total weight of the liquid, of at least 7.5 wt.-%, preferably at least 10 wt.-%, more preferably at least 12.5 wt.-%, still more preferably at least 15 wt.-%, yet more preferably at least 17.5 wt.-%, even more preferably at least 20 wt.-%, most preferably at least 22.5 wt.-%, and in particular at least 25 wt.-%.

Preferably, in step (b) the liquid contains the one or more physiologically acceptable polymers and/or copolymers at a total concentration, relative to the total weight of the liquid, of at most 47.5 wt.-%, preferably at most 45 wt.-%, more preferably at most 42.5 wt.-%, still more preferably at most 40 wt.-%, yet more preferably at most 37.5 wt.-%, even more preferably at most 35 wt.-%, most preferably at most 32.5 wt.-%, and in particular at most 30 wt.-%.

Preferably, in step (b) the liquid contains the one or more physiologically acceptable surfactants at a total concentration, relative to the total weight of the liquid, of at least 0.06 wt.-%, preferably at least 0.08 wt.-%, more preferably at least 0.10 wt.-%, still more preferably at least 0.12 wt.-%, yet more preferably at least 0.14 wt.-%, even more preferably at least 0.16 wt.-%, most preferably at least 0.18 wt.-%, and in particular at least 0.20 wt.-%.

Preferably, in step (b) the liquid contains the one or more physiologically acceptable surfactants at a total concentration, relative to the total weight of the liquid, of at most 0.75 wt.-%, preferably at most 1.0 wt.-%, more preferably at most 1.25 wt.-%, still more preferably at most 1.5 wt.-%, yet more preferably at most 1.75 wt.-%, even more preferably at most 2.0 wt.-%, most preferably at most 2.25 wt.-%, and in particular at most 2.5 wt.-%.

Preferably, in step (b) the liquid contains the one or more physiologically acceptable surfactants at a concentration within the range of from 0.0025 to 1.5% by weight. The surfactants enhance the wetting efficiency.

Preferably, in step (b) the obtained suspension or slurry contains the nanoparticles at a total concentration, relative to the total weight of the liquid, of at least 7.5 mg/mL, preferably at least 10 mg/mL, more preferably at least 12.5 mg/mL, still more preferably at least 15 mg/mL, yet more preferably at least 17.5 mg/mL, even more preferably at least 20 mg/mL, most preferably at least 22.5 mg/mL, and in particular at least 25 mg/mL.

Preferably, in step (b) the obtained suspension or slurry contains the nanoparticles at a total concentration, relative to the total weight of the liquid, of at least 75 mg/mL, preferably at least 100 mg/mL, more preferably at least 125 mg/mL, still more preferably at least 150 mg/mL, yet more preferably at least 175 mg/mL, even more preferably at least 200 mg/mL, most preferably at least 225 mg/mL, and in particular at least 250 mg/mL.

Preferably, in step (b) the obtained suspension or slurry contains the nanoparticles at a total concentration, relative to the total weight of the liquid, of at most 475 mg/mL, preferably at most 450 mg/mL, more preferably at most 425 mg/mL, still more preferably at most 400 mg/mL, yet more preferably at most 375 mg/mL, even more preferably at most 350 mg/mL, most preferably at most 325 mg/mL, and in particular at most 300 mg/mL.

Preferably, in step (b) the obtained suspension or slurry contains the nanoparticles at a total concentration, relative to the total weight of the liquid, of at most 47.5 mg/mL, preferably at most 45 mg/mL, more preferably at most 42.5 mg/mL, still more preferably at most 40 mg/mL, yet more preferably at most 37.5 mg/mL, even more preferably at most 35 mg/mL, most preferably at most 32.5 mg/mL, and in particular at most 30 mg/mL.

The nanosized Enzalutamide contained in said suspension or slurry (amorphous nanoparticles) is subsequently allowed to crystallize thereby obtaining crystalline nanosized Enzalutamide contained in said suspension or slurry (crystalline nanoparticles). Typically, 150-350 mg/mL amorphous nanosized Enzalutamide with a weight ratio of Enzalutamide to the total weight of the one or more physiologically acceptable polymers and/or copolymers amounting to about 1.0:1.0 produces desired crystalline nanosized Enzalutamide within 24 h.

The conversion of amorphous Enzalutamide into crystalline Enzalutamide typically requires some time and is preferably promoted by mixing (optional step (c)) and/or sonicating the slurry or suspension. It is contemplated that the suspension or slurry obtained in step (b) is allowed to stand for a sufficient period of time under suitable conditions. Preferably, however, the suspension or slurry obtained in step (b) is actively mixed in subsequent optional step (c).

Preferably, the conversion of amorphous Enzalutamide into crystalline Enzalutamide is allowed to take place during at least 6 hours, more preferably at least 12 hours, still more preferably for at least 16 h, even more preferably for at least 24 h. Relatively long time is preferable to achieve complete wetting and good dispersion.

Preferably, the conversion of amorphous Enzalutamide into crystalline Enzalutamide is performed at a temperature within the range of from 15° C. to 40° C., preferably from 25° C. to 35° C.

In optional step (c) of the process according to the invention, the suspension or the slurry obtained in step (b) is mixed, preferably by entraining mechanical energy.

Preferably, in step (c) mixing is performed at a temperature within the range of from 15° C. to 40° C., preferably from 25° C. to 35° C.

Preferably, in step (c) mixing is performed by stirring the suspension or the slurry.

Preferably, in step (c) mixing is performed for a duration of 2 hours to 48 hours, preferably at least 6 hours, more preferably at least 12 hours, still more preferably for at least 16 h, even more preferably for at least 24 h. Typically, in step (c) mixing is performed until the Enzalutamide is present in crystalline form.

Typically, at the end of step (b), or when the process involves step (c), at the end of step (c), the nanoparticles may be regarded as crystalline nanoparticles in the meaning of the invention, because the conversion of Enzalutamide from its amorphous state (amorphous nanoparticles, starting material) into its crystalline state (crystalline nanoparticles, product) has taken place. The optional subsequent process steps rather serve the purpose of work up, but typically do not further alter the amorphous/crystalline state of the Enzalutamide contained in the nanoparticles.

Nonetheless, the optional subsequent steps may alter the absolute and relative content of the ingredients in the composition, e.g. as a consequence of evaporation of liquid (optional step (d)) and/or as a consequence of solid/liquid separation techniques.

It is contemplated that the crystalline nanoparticles may be completely isolated from the suspension or the slurry. It is further contemplated that the thus isolated crystalline nanoparticles are purified, e.g. by washing with water.

However, as the product to be prepared by the process according to the invention is a composition comprising or essentially consisting of the crystalline nanoparticles and the one or more physiologically acceptable polymers and/or copolymers, work-up of slurry or suspension obtained at the end of step (b) or (c) preferably involves alteration of the absolute and/or relative amounts of the ingredients without completely separating the crystalline nanoparticles from the one or more physiologically acceptable polymers and/or co-polymers.

Evaporation of volatile ingredients (e.g. partial or complete removal of water in optional step (d)) will increase to absolute concentration of the crystalline nanoparticles and the one or more physiologically acceptable polymers and/or copolymers relative to the total weight of the composition, but not significantly alter the relative weight ratio of the total amount of Enzalutamide to the total amount of the one or more physiologically acceptable polymers and/or co-polymers.

Other techniques, especially solid/liquid separation techniques, will typically additionally alter the relative weight ratio of the total amount of Enzalutamide to the total amount of the one or more physiologically acceptable polymers and/or co-polymers.

Solid/liquid separation may be achieved by e.g. centrifugation, ultrafiltration, nanofiltration, or the like. While essentially the total amount of the Enzalutamide that is contained in the slurry or suspension will typically be contained in the solid phase (crystalline nanoparticles), the majority of the total amount of the one or more physiologically acceptable polymers and/or copolymers will typically be contained in the liquid phase (e.g. in aqueous solution). Thus, any techniques separating a portion of the liquid phase from the solid phase will relatively reduce the amount of the one or more physiologically acceptable polymers and/or copolymers within the composition but will not significantly affect the amount of Enzalutamide within the composition.

Depending upon the solubility of the one or more physiologically acceptable polymers and/or copolymers in the liquid and the given concentration, the one or more physiologically acceptable polymers and/or copolymers may partially precipitate such that a first portion of the one or more physiologically acceptable polymers and/or copolymers will still be present in the liquid phase in dissolved form, whereas a second portion of the one or more physiologically acceptable polymers and/or co-polymers will be present in the solid phase, i.e. together with the crystalline nanoparticles. For example, the second portion of the one or more physiologically acceptable polymers and/or copolymers may the form a solid matrix in which the crystalline nanoparticles may be embedded.

Excessive amounts of the one or more physiologically acceptable polymers and/or co-polymers as well as excessive amounts of the one or more physiologically acceptable surfactants can be removed from the suspension or slurry e.g., by filtering with hydrophilic filters, or by centrifuging and subsequently discarding the supernatant, i.e. a solution of the one or more physiologically acceptable polymers and/or copolymers and the optionally present one or more physiologically acceptable surfactants. The thus obtained residual material can be washed with solvent, e.g. water. The filtering or centrifugation step can be repeated as many times as needed.

For example, the suspension or the slurry may be centrifuged and a portion of the overhead solution (supernatant) may be decanted or otherwise removed. The remaining material, which will either be a wet solid or still a suspension or slurry, will then contain the crystalline nanoparticles at a higher concentration.

Alternatively or additionally, ultrafiltration techniques that are known to the skilled person can be performed. Centrifugal ultrafiltration devices can be used to purify, wash, and concentrate the crystalline nanoparticles based on size. Centrifugal ultrafiltration units are commercially available, e.g. Amicon® Ultra (Merck KGaA) and Centricon® Plus (Merck Millipore). Purification systems are also commercially available, e.g. Amicon® Pro (Merck KGaA). Separation and concentration of molecules during ultrafiltration is based on size exclusion. The great majority of biomolecules have a molecular weight less than 500,000 Da, and the crystalline nanoparticles fit nicely into this category. Suitable filter systems are offered with membrane nominal molecular weight limits (NMWLs) of 3,000, 10,000, 30,000, 50,000 and 100,000 Da. To retain the crystalline nanoparticles, the molecular weight cut-off of the filter membrane needs to be smaller than the crystalline nanoparticle, but large enough to allow smaller components to filter through.

In optional step (d) of the process according to the invention, the suspension or slurry obtained in step (b), the mixed suspension or slurry obtained in step (c), or the subsequently concentrated suspension or slurry is dried thereby yielding a residual composition, e.g. a solid dry material or a solid but still wet material. Drying typically involves evaporation of volatile liquid constituents.

Drying may be complete or partial. Thus, for the purpose of the specification, drying may involve evaporation of essentially the total quantity of volatile liquid constituents (complete) or only of a part thereof (partial).

Partial drying is preferred, as the thus obtained residual composition still contains residual amounts of liquid (e.g. water) which may subsequently be used as granulation liquid for preparing pharmaceutical formulations or pharmaceutical dosage forms from the composition.

According to other preferred embodiments, the suspension or the slurry is dried to give a solid residual composition comprising crystallized nanosized Enzalutamide (crystalline nanoparticles), the one or more physiologically acceptable polymers and/or copolymers, and optionally the one or more physiologically acceptable surfactants.

In the solid state, i.e. after removal of essentially the total amount of volatile liquid constituents, the crystalline nanoparticles according to the invention, the precipitated one or more physiologically acceptable polymers and/or copolymers, and the optionally present precipitated one or more physiologically acceptable surfactants may be associated with one another. Alternatively, the one or more physiologically acceptable polymers and/or copolymers, and the optionally present one or more physiologically acceptable surfactants may independently of one another be divided in a first fraction and a second fraction, wherein the first fraction is associated with the crystalline nanoparticles according to the invention, and wherein the second fraction is not associated with the crystalline nanoparticles according to the invention.

The same applies to association in semiliquid states where residual amounts of liquid are still present which, however, are not enough to dissolve the total amount of the one or more physiologically acceptable polymers and/or copolymers, and/or the total amount of the optionally present one or more physiologically acceptable surfactants thereby inducing their (partial) precipitation.

Association may be of any kind. For example, as mentioned above, the crystalline nanoparticles may be embedded in a matrix comprising the one or more physiologically acceptable polymers and/or copolymers, and the optionally present one or more physiologically acceptable surfactants. It is also possible that the surface of the crystalline nanoparticles is completely or partially coated with the one or more physiologically acceptable polymers and/or copolymers, and the optionally present one or more physiologically acceptable surfactants.

When the process according to the invention does not involve solid/liquid separation techniques, the total content of
the Enzalutamide;
the one or more physiologically acceptable polymers and/or copolymers; and
the one or more physiologically acceptable surfactants;
as contained in the suspension or slurry obtained in step (b) remains essentially constant and thus is likewise contained in the dried residual composition.

Drying is preferably carried out in an oven, preferably at a temperature within the range of from 20 to 40° C., more preferably about 30° C., for e.g. 24 h, or until the remaining water content is ≤5 wt.-%.

Drying can be done by using methods known in the art. Exemplary drying methods comprise heating, evaporating, vacuum drying, using a fluidized bed dryer, spray drying, and freeze drying.

In optional step (e) of the process according to the invention, the crystalline nanoparticles are crushed or grinded, e.g. with a suitable mill. Crushing or grinding the dried residual composition yields a powder ready for tableting.

Crystalline nanoparticles are preferably obtained by drying in step (d) followed by crushing/grinding in step (e).

Another aspect of the invention relates to a composition which is obtainable or obtained by the process according to the invention as described above.

Another aspect of the invention relates to a pharmaceutical formulation comprising the composition according to the invention as described above and one or more pharmaceutical excipients. Said one or more pharmaceutical excipients typically differ from the one or more physiologically acceptable polymers and/or copolymers and the one or more optional physiologically acceptable surfactants that are already contained in the composition according to the invention as described above.

It is contemplated that additional amounts of one or more pharmaceutical excipients that are already contained in the composition according to the invention (e.g. polymer or copolymer) may be added as pharmaceutical excipient to the pharmaceutical formulation according to the invention, such that a first portion thereof is already contained in the composition and the remainder thereof is added when the pharmaceutical formulation is prepared from the composition. In a preferred embodiment, however, the pharmaceutical excipients that are contained in the composition differ from the pharmaceutical excipients that are added when the pharmaceutical formulation is prepared from the composition.

Preferably, the pharmaceutical excipients are selected from the group consisting of fillers, binders, disintegrants, surfactants, lubricants, glidants, retardant polymers and any combination thereof.

Examples of fillers (diluents) include but are not limited to starch, lactose, xylitol, sorbitol, confectioner's sugar, compressible sugar, dextrates, dextrin, dextrose, fructose, lactitol, mannitol, sucrose, talc, micro crystalline cellulose, calcium carbonate, calcium phosphate dibasic or tribasic, dicalcium phosphate dehydrate, calcium sulfate, and the like. Fillers typically represent from 20 wt.-% to 80 wt.-% of the pharmaceutical formulation.

Examples of binders include but are not limited to starches such as potato starch, wheat starch, corn starch; microcrystalline cellulose; celluloses such as hydroxypropyl cellulose, hydroxyethyl cellulose, hydroxypropylmethylcellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, and the like. Binders typically represent up to 10 wt.-% of the pharmaceutical formulation.

Examples of disintegrants include, but are not limited to alginic acid, methacrylic acid DVB, cross-linked PVP, microcrystalline cellulose, sodium croscarmellose, crospovidone, polacrilin potassium, sodium starch glycolate, starch, including corn or maize starch, pregelatinized starch and the like. Disintegrant(s) typically represent up to 20 wt.-% of the pharmaceutical formulation.

Examples of surfactants have already been described above in connection with the pharmaceutical excipients that are preferably contained in the crystalline nanoparticles. The same surfactants are principally also useful. Surfactant(s) typically represent up to 5.0 wt.-% of the pharmaceutical formulation.

Examples of lubricants include, but are not limited to magnesium stearate, aluminum stearate, calcium stearate, zinc stearate, stearic acid, polyethylene glycol, glyceryl behenate, mineral oil, sodium stearyl fumarate, talc, hydrogenated vegetable oil and the like. Lubricants typically represent from 0.2 wt.-% to 5.0 wt.-% of the pharmaceutical formulation.

Examples of glidants include but are not limited to silicon dioxide, colloidal anhydrous silica, magnesium trisilicate, tribasic calcium phosphate, calcium silicate, magnesium silicate, colloidal silicon dioxide, powdered cellulose, starch, talc, and the like. Glidants typically represent from 0.01 wt.-% to 0.3 wt.-% of the pharmaceutical formulation.

Examples of retardant polymers include but are not limited to cellulose derivatives such as cellulose ethers or cellulose esters; guar and guar derivatives; pectin; carrageenan; xanthan gum; locust bean gum; agar; algin and its derivatives, gellan gum, acacia, starch and modified starches; and synthetic polymers; including but not limited to homo- and co-polymers of carboxyvinyl monomers, homo- and co-polymers of acrylates or methacrylate monomers, homo- and co-polymers of oxyethylene, or oxypropylene monomers; or any combination of the foregoing.

The weight content of the Enzalutamide in the pharmaceutical formulation is not particularly limited. Preferably, the weight content of the Enzalutamide is at least 1.0 wt.-%, preferably at least 2.5 wt.-%, more preferably at least 5.0 wt.-%, in each case relative to the total weight of the pharmaceutical formulation. Preferably, the weight content of the Enzalutamide is at least 10 wt.-%, preferably at least 15 wt.-%, more preferably at least 20 wt.-%, still more preferably at least 25 wt.-%, yet more preferably at least 30 wt.-%, even more preferably at least 35 wt.-%, still more preferably at least 40 wt.-%, most preferably at least 45 wt.-%, and in particular at least 50 wt.-%, in each case relative to the total weight of the pharmaceutical formulation.

In particularly preferred embodiments, the pharmaceutical dosage form according to the invention comprises one or more excipients selected from fillers, disintegrants, glidants and lubricants.

Preferably, the total content of
the nanoparticles comprising or essentially consisting of Enzalutamide in crystalline form;
the one or more physiologically acceptable polymers and/or copolymers; and
the optional one or more physiologically acceptable surfactants
present in the pharmaceutical dosage form amounts to at least 20 wt.-%, preferably at least 22.5 wt.-%, more preferably at least 25 wt.-%, still more preferably at least 27.5 wt.-%, yet more preferably at least 30 wt.-%, even more preferably at least 32.5 wt.-%, most preferably at least 35 wt.-%, and in particular at least 37.5 wt.-%, in each case relative to the total weight of the pharmaceutical dosage form.

Preferably, the pharmaceutical dosage form according to the invention contains one or more fillers; preferably microcrystalline cellulose, a cellulose ether, or a mixture thereof.

Preferably, the total content of the one or more fillers amounts to at least 30 wt.-%, preferably at least 32.5 wt.-%, more preferably at least 30 wt.-%, still more preferably at least 32.5 wt.-%, yet more preferably at least 35 wt.-%, even more preferably at least 37.5 wt.-%, most preferably at least 40 wt.-%, and in particular at least 42.5 wt.-%, in each case relative to the total weight of the pharmaceutical dosage form.

In preferred embodiments, the pharmaceutical dosage form according to the invention contains one or more disintegrants; preferably croscarmellose sodium.

Preferably, the total content of the one or more disintegrants amounts to at least 0.5 wt.-%, preferably at least 1.0 wt.-%, more preferably at least 1.5 wt.-%, still more preferably at least 2.5 wt.-%, in each case relative to the total weight of the pharmaceutical dosage form.

Preferably, the pharmaceutical dosage form according to the invention contains one or more lubricants; preferably magnesium stearate, silica, or a mixture thereof.

Preferably, the pharmaceutical dosage form according to the invention contains no binder other than the one or more physiologically acceptable polymers and/or co-polymers.

Preferably, the pharmaceutical dosage form according to the invention comprises or essentially consists of an intragranular phase and an extragranular phase.

Preferably, essentially the total content of
the nanoparticles comprising or essentially consisting of Enzalutamide in crystalline form;
the one or more physiologically acceptable polymers and/or copolymers; and
the optional one or more physiologically acceptable surfactants
present in the pharmaceutical dosage form is contained in the intragranular phase.

In preferred embodiments, the pharmaceutical dosage form according to the invention contains a first portion of disintegrant in the intragranular phase and a second portion of disintegrant in the extragranular phase.

Preferably, the weight content of the intragranular phase amounts to at least 60 wt.-%, preferably at least 65 wt.-%, more preferably at least 70 wt.-%, still more preferably at least 75 wt.-%, yet more preferably at least 80 wt.-%, even more preferably at least 85 wt.-%, most preferably at least 90 wt.-%, and in particular at least 95 wt.-%, in each case relative to the total weight of the pharmaceutical dosage form.

Another aspect of the invention relates to a pharmaceutical dosage form comprising the composition according to the invention as described above or the pharmaceutical formulation according to the invention as described above.

Preferably, the pharmaceutical dosage form is selected from tablets, micro tablets, capsules, powders, granules, suspensions, emulsions.

In a preferred embodiment, the pharmaceutical dosage form according to the invention is film-coated tablet.

The total weight of the pharmaceutical dosage form according to the invention is not particularly limited. However, as far as oral dosage forms are concerned, the size should preferably not exceed a certain limit for ease of swallowing and patient compliance.

Preferably, the pharmaceutical dosage form has a total weight of not more than 1000 mg, preferably not more than 950 mg, more preferably not more than 900 mg, still more preferably not more than 850 mg, yet more preferably not more than 800 mg, even more preferably not more than 750 mg, most preferably not more than 700 mg, and in particular not more than 650 mg.

In preferred embodiments, the pharmaceutical dosage form according to the invention contains the Enzalutamide at a dose within the range of 30±15 mg, or 40±20 mg, or 60±30 mg, or 80±40 mg, or 120±60 mg, or 150±75 mg, or 160±80 mg, or 200±80 mg, or 240±120 mg, or 300±150 mg, or 360±180 mg, in each case expressed as weight equivalent of the non-salt form of Enzalutamide. Preferably, the dose of Enzalutamide is 120 mg or 160 mg.

Another aspect of the invention relates to a process for the preparation of the pharmaceutical dosage form according to the invention as described above comprising the steps of
(i) providing a composition according to the invention as described above;
(ii) granulating, preferably wet-granulating the composition with one or more pharmaceutical excipients; and
(iii) compressing the granulate.

Preferably, the process for the preparation of the pharmaceutical dosage form comprises the process for the preparation of the composition according to the invention as described above.

Another aspect of the invention relates to the pharmaceutical dosage form according to the invention as described above for use in the treatment of a hyperproliferative disorder. Another aspect of the invention relates to a method of treating a hyperproliferative disorder comprising administering the pharmaceutical dosage form according to the invention as described above to a subject in need thereof. Another aspect of the invention relates to the use of Enzalutamide for the manufacture of a pharmaceutical dosage form according to the invention as described above for treating a hyperproliferative disorder.

Preferably, the hyperproliferative disorder is selected from the group consisting of benign prostatic hyperplasia, prostate cancer, breast cancer, and ovarian cancer. Preferably, the hyperproliferative disorder is prostate cancer selected from hormone-refractory prostate cancer and hormone-sensitive prostate cancer.

Preferably, the pharmaceutical dosage form according to the invention is administered orally.

Preferably, the pharmaceutical dosage form according to the invention is administered once daily or twice daily; preferably once daily; in each case optionally involving simultaneous administration of a plurality of pharmaceutical dosage forms. In this regard “simultaneous administration” means that more than one pharmaceutical dosage form is taken by a subject within a relatively short period of time, e.g. within 10 minutes, preferably within 5 minutes.

In a preferred embodiment, the pharmaceutical dosage form according to the invention is orally administered after a meal. In another preferred embodiment, the pharmaceutical dosage form according to the invention is orally administered before a meal.

The following examples further illustrate the invention but are not to be construed as limiting its scope.

### Materials and Methods

The amorphous nanosized Enzalutamide was prepared from bulk Enzalutamide using the process disclosed in U.S. Pat. No. 10,098,842.

SEM images were captured using the Zeiss Sigma 300 VP SEM instruments. Samples were dispersed into water and filtered with 0.1 μm filter. Filters were dried, transferred to SEM sample holders and coated with a 5 nm thick layer of platinum.

XRPD measurements were carried out using the Malvern PANalytical Empyrean X-ray diffractometer equipped with a Cu Kα (1.54 Å) source, MultiCore optics and a solid-state PIXcel3D detector. By using Kapton tape the samples were attached onto aluminum or polycrystalline silicon sample holders. Dried slurries were measured without further sample preparation under Kapton tape and suspensions were filtered, dried, and filters were attached with double sided tape. The samples were measured in the reflection geometry in a spinning measurement stage. The measurement range was 5-40 (° 20). The step size and time per step values were varied depending on the counts per second obtained.

Dynamic light scattering (DLS) measurements were performed with Malvern Zetasizer. Slurries or dried powder were redispersed into water or 0.1% HPMC (aq), stirred and measured after the sample was completely dispersed. Backscattering measurement setup was used, and CUMULANTS-algorithm was used to obtain average particle diameter (Z-average) and polydispersity index (PI).

### Example 1—Preparing Amorphous Nanoparticles of Enzalutamide

Amorphous nanoparticles of Enzalutamide were prepared from solutions of Enzalutamide in supercritical CO₂ by Controlled Expansion of Supercritical Solutions (CESS™) (U.S. Pat. No. 10,098,842, in analogy to Example 1 thereof).

The CESS™ process was performed by using an apparatus comprising a pressure vessel, a tube, and a depressurization vessel connected to one another in serial arrangement.

### Comparative Example—Crystallization of Nanosized Amorphous Enzalutamide from Water

Amorphous enzalutamide nanoparticles obtained in accordance with Example 1 were weighted into small glass vial. Deionized (DI) water was added to the amount that leads to 5 mg/ml Enzalutamide content. The mixture was mixed with magnetic stirrer and ultrasonicated until all Enzalutamide was completely surrounded by water (wetted) and the stirring was continued at least an hour. SEM and XRD samples were prepared as described above. SEM images showed formation of large (>2 μm) Enzalutamide crystals.

### Example 2—Suspending Amorphous Particles of Enzalutamide in Polymer Solutions

Amorphous nanoparticles of Enzalutamide obtained in accordance with Example 1 were weighted into small glass vials. Aqueous polymer solutions were added to an amount leading to 25 mg/ml Enzalutamide content. The mixtures were mixed with magnetic stirrer and ultrasonicated until all Enzalutamide was completely surrounded by water (wetted). The stirring was continued for 16-24 h. SEM and XRPD samples were prepared as described above. Results are summarized in the table here below:

| Ex. | Aqueous solution | grade^{a} | comment |
|---|---|---|---|
| 2-1 | pure water | 1 | >1 μm sized crystals |
| 2-2 | HPMC, SLS, Tween 80 | 1 | nanoscale but amorphous |
| 2-3 | PVP-VA, with SLS, Tween 80 | 5 | small nanocrystals |
| 2-4 | Poloxamer 407 | 3 | crystalline but mainly |
| | | | microscale |
| 2-5 | Poloxamer 407, Tween 80, | 3 | crystalline but mainly |
| | TPGS | | microscale |
| 2-6 | methyl cellulose, Tween 80 | 2 | nanoscale but low |
| | | | crystallinity |

^{a}grade: 1-5, 5 = nanocrystals, 1 = large crystals or no crystallization.

As demonstrated by the above experimental data, the best nanocrystals of Enzalutamide were obtained when the crystallization was performed using aqueous solution comprising PVPVA. It was surprisingly found that only under the conditions of Example 2-3 Enzalutamide crystallizes, i.e. is transformed from its original amorphous state into a crystalline state, and maintains a particle size in the nanoscale. PVP-VA facilitates formation of nanoflakes that are at least partly crystalline. Crystallization from aqueous suspension/slurry comprising PVP-VA in aqueous solution reduces the particle size significantly.

When PVP-VA was replaced by other polymers, formation of the desired crystalline Enzalutamide nanoparticles was reduced significantly. The corresponding Example 2-2 with HPMC instead of poly(vinylpyrrolidone vinylacetate) copolymer did not induce crystallization of Enzalutamide.

In additional experiments, the concentration of the amorphous nanoparticles of Enzalutamide and the concentration of the PVP-VA were varied in order to investigate the effect on crystal size. Suspensions/slurries of 25 mg/mL Enzalutamide and 5 wt.-% PVP-VA (high water content) yielded flakes as well as large crystals, whereas slurries of 250 mg/mL Enzalutamide and 25 wt.-% PVP-VA (low water content) yielded flakes.

Electron micrographs revealed that increasing the content of PVP-VA in solution leads to a reduction of particle size. Electron micrographs for solutions of PVP-VA at concentrations of 1 wt.-%, 2.5 wt.-%, 5 wt.-% and 10 wt.-% are shown in FIGS. 1 to 4 (A at 25,000 fold magnification, B at 5,000 fold magnification).

Further, XRPD analysis revealed that decreasing the content of PVP-VA in solution favors crystallinity (see FIG. 5).

### Example 3—Varying Mixing Conditions

In accordance with Example 2, amorphous nanoparticles of Enzalutamide were suspended in aqueous solutions of poly(vinylpyrrolidone vinylacetate) copolymer (PVP-VA, copovidone). Solutions additionally contained 0.2 wt.-% sodium lauryl sulfate (SLS). The thus obtained suspensions/slurries were mixed under various conditions and the properties of the thus obtained crystalline nanoparticles were investigated.

It was observed that extended mixing times break up agglomerates, whereas increased concentrations of PVP-VA speed up the process (see FIG. 6, z-average particle sizes in nm).

Similar results could be achieved by bath sonication (see FIG. 7, z-average particle sizes in nm).

Additional suspension/slurry experiments were performed at the following ratios/weight percentages of Enzalutamide: PVP-VA: (a) 15:15%, (b) 20:20%, (c) 20:10%, (d) 20:30%, (e) 25:25%, (f) 30:30%, and (g) 35:35%.

The properties of the thus obtained crystalline nanoparticles were investigated by electron microscopy. For the ratio (f) 30:30%, crystallinity was observed by XRPD as a function of mixing time. FIG. 8 shows XRPD intensity counts of crystalline nanoparticles of Enzalutamide obtained from suspensions/slurries in aqueous solutions of PVA-VA at concentrations of 30 wt.-% Enzalutamide and 30 wt.-% PVP-VA at different mixing times.

It was revealed on the basis of the electron micrographs that optimal results can be achieved at a relative weight ratio of Enzalutamide: PVP-VA of about 1:1, a concentration of PVP-VA in aqueous solution of about 20 to 30 wt.-%, and at mixing tomes of about 6 to 24 hours.

### Example 4—Preparation of Crystalline Nanoparticles of Enzalutamide with and without Surfactant

Amorphous nanoparticles of Enzalutamide in accordance with Example 1 were suspended in aqueous solutions of PVP-VA at concentrations of 20 wt.-%, in the absence and in the presence of 0.2 wt.-% SLS in accordance with Examples 2 and 3.

FIG. 9 shows z-average particle sizes in nm, FIG. 10 corresponding XRPD intensity counts of the thus obtained wet and dry particles, in the absence and in the presence of SLS.

### Example 5—Comparing Nanosuspensions In Vivo

### Preparation of the Suspensions:

180 mg of dried nanocrystals generated by the controlled crystallization process was filled into each vial and 18 mL (4×4.5 mL) of the various suspension vehicles were added 45 min-1.5 h prior dosing. To assure the suspensions were well dispersed the suspensions were mixed using a magnetic stirrer until administration. Enzalutamide concentration was 5 mg/mL. The Xtandi® suspension was prepared in 1% MC

### Pharmacokinetic Study Plan in Rats:

Topic

| | |
|---|---|
| Study type | Pharmacokinetic |
| Rat population | Sprague Dawley, male, weight ~300-350 g, 8.5 weeks |
| | old |
| Number of rats | 5-6 |
| per group | |
| Way of | Oral suspension, dose volume: 1.5 ml. |
| administration | |
| Dose | 20 mg/kg |
| Sampling | 10, 30, 60, 90, 150, 210, 270, 360, 450, 720, 1440, 2880 |
| points [min] | |

### Results:

Suspensions of enzalutamide nanocrystals showed similar plasma concentration vs. time profiles compared to the 1% MC Xtandi® suspension; AUC₀₋ₗₐₛₜ, Cₘₐₓ, or Tₘₐₓ were similar. Pharmacokinetic parameters of nanocrystals in different suspension vehicles compared to Xtandi® ASD formulation in 1% MC suspension are compiled in the table here below:

| | | AUC₀₋ₗₐₛₜ | Cₘₐₓ | Tₘₐₓ |
|---|---|---|---|---|
| | Group | (h · μg/ml) | (μg/ml) | (h) |
| — | Xtandi ® in MC | 208 ± 47 | 12.1 ± 2.3 | 3.8 ± 1.3 |
| - - - | Nanocrystals in 1% HPMC- | 189 ± 22 | 10.5 ± 1.5 | 3.9 ± 1.2 |
| | E + 0.1% SLS | | | |
| — | Nanocrystals in 1% SLS | 193 ± 35 | 9.9 ± 1.0 | 3.5 ± 0.6 |

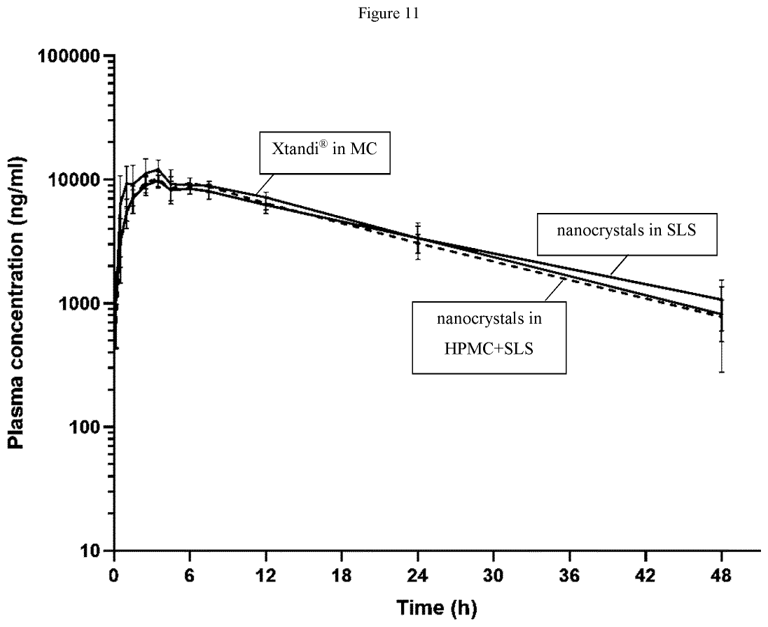

FIG. 11 shows the plasma concentration time curves.

Conclusions from In Vivo Study:

Nanocrystalline formulations reached equivalent exposure as the reference product Xtandi® in a pharmacokinetic in vivo study in rats. The various suspension formulations of Enzalutamide showed similar performance.

### Example 6—Tablets

Three formulations were manufactured by using the wet granulation technique. A drug loading of 20% w/w was used and tablet compression parameters (e.g., die cavity height, compression force, ejection force, strokes/min) were kept constant to investigate the impact of excipients on the tablet properties.

Compressed tablets were manufactured by a process comprising the following steps:
1. Controlled crystallization: Enzalutamide and copovidone slurry was prepared initially and considered as preliminary/starting material for further wet granulation process.
2. Wet Blending: The dry powder excipients (filler, binder, disintegrant etc.) were added to the intermediate wet mass during the continuous mixing by using an overhead stirrer.
3. Wet granulation: Mixing was continued with the stirrer to allow proper wetting and cohesiveness and to provide a homogenous mix of the active and the excipients. The wet mass was passed through a larger screen sieve and collected in a suitable container for the drying process. Prior to the drying process, granules were layered/spread properly in a container to allow a uniform drying process
4. Drying: The granules were dried at an inlet temperature of 32±5° C. (to an LOD below 5%) and at 10% relative humidity in a tray dryer/hot air oven.
5. Grinding and sieving: The dry granules were milled with mortar and pastel. Milled granules were passed through different sieves to create different size fractions. Required quantities of different granule fractions were weighed individually.
6. Lubrication: Granules were blended in a separate container with extra granular excipients, magnesium stearate and Aerosil®.
7. Compression: The final blend was manually filled into the die cavity. Tablets were compressed at predefined process parameters.
8. Dedusting and storage: Compressed tablets were collected and dedusted manually and stored in a closed glass vial until further investigations.

The tree different formulations were evaluated to study the impact of excipient concentrations on the tablet quality. The composition of the formulations is compiled in the table here below:

| | | | 6-1 | | 6-2 | | 6-3 | |
|---|---|---|---|---|---|---|---|---|
| ingredients | | function | mg | wt. % | mg | wt. % | mg | wt. % |
| intra | Enzalutamide | active ingredient | 40 | 20 | 40 | 19.8 | 160 | 20 |
| | PVP-VA | copolymer/binder | 40 | 20 | 40 | 19.8 | 160 | 20 |
| | MCC | filler | 100 | 50 | 90 | 44.55 | 432 | 54 |
| | HPMC-E | filler/stabilizer | 10 | 5 | — | | — | |
| | croscarmellose Na | disintegrant | 8 | 4 | 30 | 14.85 | 20 | 2.5 |
| extra | croscarmellose Na | disintegrant | — | — | — | — | 20 | 2.5 |
| | Mg stearate | lubricant | 1 | 0.5 | 1 | 0.5 | 4 | 0.5 |
| | SiO₂ | glidant | 1 | 0.5 | 1 | 0.5 | 4 | 0.5 |

| | | | | | | |
|---|---|---|---|---|---|---|
| total | 200 | 100 | 202 | 100 | 800 | 100 |

intra = intragranular;
extra = extragranular

No change in tablet property was observed when tablets were prepared with or without additional binding agent-HPMC. It was concluded that the excipient concentration and type have only small impact on tablet hardness and disintegration time.

1. A composition comprising
nanoparticles comprising or essentially consisting of Enzalutamide in crystalline form;
one or more physiologically acceptable polymers or copolymers; and
optionally, one or more physiologically acceptable surfactants;
wherein the nanoparticles have a z-average particle size of at most 800 nm and at least 10 nm; and
wherein the relative weight ratio of the total amount of Enzalutamide to the total amount of the one or more physiologically acceptable polymers or copolymers is within the range of from 5.0:1.0 to 1.0:5.0.

2.-4. (canceled)

5. The composition according to any of the preceding claims, wherein the one or more physiologically acceptable polymers or copolymers comprise a vinylpyrrolidone vinylacetate copolymer, and
wherein the vinylpyrrolidone vinylacetate copolymer is a copolymer of 1-vinyl-2-pyrrolidone and vinylacetate in the mass proportion of about 3:2 or
wherein the vinylpyrrolidone vinylacetate copolymer contains at least 35.0 wt.-% and at most 42.0 wt.-% of vinylacetate, calculated on the dried basis.

6.-7. (canceled)

8. The composition of claim 1, wherein the total content of the one or more physiologically acceptable polymers and/or co-polymers, relative to the total dry solids content of the composition, is at least 7.5 wt.-% and is at most 90 wt.-%.

9.-12. (canceled)

13. The composition of claim 1, wherein the content of Enzalutamide, relative to the total dry solids content of the composition, is at least 7.5 wt.-%, and is at most 90 wt.-%.

14.-16. (canceled)

17. The composition of claim 1, wherein the relative weight ratio of the total amount of Enzalutamide to the total amount of the one or more physiologically acceptable polymers and/or copolymers is within the range of from 4.5:1.0 to 1.0:4.5.

18. The composition of claim 1, wherein the one or more physiologically acceptable surfactants comprise a surfactant selected from the group consisting of (i) alkyl sulfate salts; preferably selected from sodium lauryl sulfate (sodium dodecyl sulfate), sodium cetyl sulfate, sodium cetyl stearyl sulfate, sodium stearyl sulfate, sodium dioctylsulfosuccinate; and the corresponding potassium or calcium salts thereof; (ii) fatty acid salts; preferably selected from stearic acid salts, oleic acid salts; (iii) salts of cholic acid; preferably selected from sodium deoxycholate, sodium glycocholate, sodium taurocholate and the corresponding potassium or ammonium salts.

19. The composition of claim 9, wherein the one or more physiologically acceptable surfactants comprise a surfactant selected from the group consisting of sodium lauryl sulfate (SLS) Tween 80, Tween 20, dioctyl sulfosuccinate sodium salt (DOSS), and tocofersolan (TPGS).

20. (canceled)

21. The composition of claim 1, wherein the total content of the one or more physiologically acceptable surfactants, relative to the total weight of the composition, is at most 7.0 wt.-%.

22. (canceled)

23. The composition of claim 1, wherein the composition is a suspension or a slurry comprising (i) a solid phase comprising or essentially consisting of the nanoparticles and (ii) a liquid phase, wherein the total content of the one or more surfactants in the liquid phase is within the range from 0.0025 to 1.5 wt.-%.

24. The composition of claim 1, wherein the nanoparticles are nanoflakes.

25.-28. (canceled)

29. The composition of claim 1, which comprises:
nanoparticles comprising or essentially consisting of Enzalutamide in crystalline form;
a vinylpyrrolidone vinylacetate copolymer; and
optionally, sodium lauryl sulfate;
wherein the nanoparticles have a z-average particle size of at most 700 nm; and
wherein the relative weight ratio of the total amount of Enzalutamide to the vinylpyrrolidone vinylacetate copolymer is within the range of from 4.0:1.0 to 1.0:4.0.

30.-32. (canceled)

33. The composition of claim 1, which comprises (i) a solid phase, preferably comprising or essentially consisting of the nanoparticles; and (ii) a liquid phase, preferably wherein at least a portion of the one or more physiologically acceptable polymers and/or copolymers is dissolved in the liquid phase.

34. (canceled)

35. The composition according to claim 33, which has a water content of at least 5.0 wt.-% relative to the total weight of the composition (suspension or slurry).

36.-38. (canceled)

39. The composition of claim 33, which contains the nanoparticles at a total concentration, relative to the total weight of the composition (slurry or suspension), of at least 7.5 mg/mL.

40.-43. (canceled)

44. A process for the preparation of the nanoparticles of claim 1, the process comprising the steps of
(a) providing nanoparticles comprising Enzalutamide in amorphous form;
(b) contacting the nanoparticles provided in step (a) with the one or more physiologically acceptable polymers and/or copolymers in a liquid thereby obtaining a suspension or a slurry;
(c) optionally, mixing the suspension or the slurry;
(d) optionally, drying the suspension or the slurry thereby obtaining a residual composition; and
(e) optionally, crushing the residual composition.

45. The process according to claim 4, wherein step (a) involves dissolving Enzalutamide in supercritical CO₂.

46. The process of claim 44, wherein the amorphous nanoparticles provided in step (a) have a z-average particle size of at most 600 nm and of at least 60 nm.

47. (canceled)

48. The process of claim 44, wherein the amorphous nanoparticles provided in step (a) have a particle size distribution characterized by a Dᵥ90 value of at most 1000 nm and or at least 50 nm.

49. (canceled)

50. The process of claim 44, wherein in step (b) the liquid, preferably aqueous solution, contains the one or more physiologically acceptable polymers or copolymers at a concentration within the range of from 0.2 to 40 wt.-% by weight relative to the total weight of the liquid.

51. (canceled)

52. (canceled)

53. The process of claim 44, wherein in step (b) the liquid contains the one or more physiologically acceptable surfactants at a total concentration, relative to the total weight of the liquid, of at least 0.06 wt.-% and at most 2.5 wt-%.

54. (canceled)

55. (canceled)

56. The process of claim 44, wherein in step (b) the obtained suspension or slurry contains the nanoparticles at a total concentration, relative to the total weight of the liquid, of at least 7.5 mg/mL and at most 475 mg/mL.

57.-59. (canceled)

60. The process of claim 44, wherein in step (c) mixing is performed at a temperature within the range of from 15° C. to 40° C., preferably from 25° C. to 35° C., or wherein in step (c) mixing is performed by stirring the suspension or the slurry, or wherein in step (c) mixing is performed for a duration of 2 hours to 48 hours.

61.-63. (canceled)

64. A pharmaceutical dosage form for oral administration comprising the composition of claim 1 and one or more excipients selected from fillers, disintegrants, glidants and lubricants.

65. The pharmaceutical dosage form of claim 64, which has been prepared by a process involving wet granulation.

66. The pharmaceutical dosage form of claim 64, which is selected from tablets, film coated tablets, micro tablets, capsules, powders, granules, suspensions, and emulsions.

67. (canceled)

68. The pharmaceutical dosage form claim 64, which has a total weight of not more than 1000 mg.

69. The pharmaceutical dosage form of claim 64, which contains the Enzalutamide at a dose within the range of 30±15 mg expressed as weight equivalent of the non-salt form of Enzalutamide.

70. (canceled)

71. (canceled)

72. The pharmaceutical dosage form of claim 64, which contains one or more fillers; preferably microcrystalline cellulose, a cellulose ether, or a mixture thereof, or wherein the total content of the one or more fillers is at least 30 wt.-% relative to the total weight of the pharmaceutical dosage form.

73. (canceled)

74. The pharmaceutical dosage form of claim 64, which contains one or more disintegrants; or the disintegrant comprises croscarmellose sodium, or the total content of one or more disintegrants is at least 0.5 wt.-% relative to the total weight of the pharmaceutical dosage form.

75. (canceled)

76. The pharmaceutical dosage form of claim 64, which comprises magnesium stearate, silica, or a mixture thereof, or which comprises an intragranular phase and an extragranular phase.

77.-87. (canceled)
